(19) United States
(12) Patent Application Publication
(10) Pub. No.: US 2013/0165450 A1
(43) Pub. Date: Jun. 27, 2013

(54) Novel Thiazol-Carboximide Derivatives as PDK1 Inhibitors

(71) Applicants: Hon-Chung Tsui, Shanghai (CN); Sunil Paliwal, Monroe Township, NJ (US); Thierry Oliver Fischmann, Scotch Plains, NJ (US)

(72) Inventors: Hon-Chung Tsui, Shanghai (CN); Sunil Paliwal, Monroe Township, NJ (US); Thierry Oliver Fischmann, Scotch Plains, NJ (US)

(21) Appl. No.: 13/820,913

(22) PCT Filed: Sep. 9, 2011
(86) PCT No.: PCT/US2011/050910

§ 371 Date: Mar. 5, 2013

Related U.S. Application Data
(60) Provisional application No. 61/382,622, filed on Sep. 14, 2010.

(51) Int. Cl. C07D 417/14; C07D 491/113; C07D 471/10

(52) U.S. Cl. CPC C07D 417/14; C07D 471/10; C07D 491/113
USPC 514/254.02; 514/278; 514/316; 514/326; 544/369; 546/16; 546/19; 546/187; 546/209

(57) ABSTRACT

This invention relates to certain thiazole carboxamide derivatives of Formula (I) as inhibitors of 3-phosphoinositide-dependent protein kinase (PDK-1). The compounds can be useful in inhibiting the proliferation of cancer cells, and other aberrant conditions where the PDK-1 signaling pathway is overstimulated.

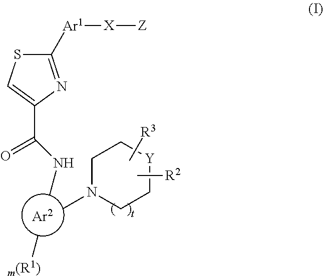

### FIELD OF THE INVENTION

This invention relates to certain thiazole carboxamide derivatives of Formula (I) as inhibitors of 3-phosphoinositide-dependent protein kinase (PDK-1). The compounds are useful in inhibiting the proliferation of cancer cells, and other aberrant conditions where the PDK-1 signaling pathway is overstimulated.

### BACKGROUND OF THE INVENTION

Certain kinases that belong to the serine/threonine kinase family are located intracellularly and are involved in the transmission of biochemical signals such as those that affect cell proliferation and survival. One such serine/threonine kinase is PDK1, which is a regulator of at least 23 protein kinases that belong to the AGC kinase family (cAMP-dependent, cGMP-dependent, and protein kinase C). Signal transduction pathways downstream of PDK1 include the serine/threonine kinases protein kinase B (PKB/Akt), p70 ribosomal S6 kinase (p70S6K1), serum- and glucocorticoid-induced protein kinase (SGK), p90 ribosomal S6 kinase (RSK), and protein kinase C(PKC). Peifer et al., ChemMed Chem 3, 1810-1838 (2008).

The binding of growth factors to the cell surface receptors activates phosphoinositide-3 kinase (PI3K), which phosphorylates the substrate, phosphoinositidylinositol-4,5-triphosphate (PIP2) to form the second messenger, phosphoinositidylinositol-3,4,5-triphosphate (PIP3). PIP3 binds to both PDK1 and PKB/Akt, which are believed to co-localize at the cell membrane as a consequence. In addition to its interaction with PKB/Akt, PDK1 also phosphorylates and activates p70S6K1, SGK, RSK and PKC, which influences cell growth, proliferation, and survival, and regulates metabolism. Bayascas, J. R., Cell Cycle, 7, 2978-2982 (2008).

Cancer cells of common human tumor types, including breast, lung, gastric, prostate, haematological and ovarian cancers, have gene mutations that result in abnormally high levels of PIP3. High levels of PIP3 cause overstimulation of PDK1 which result in constitutive activation the members of the AGC kinase family. As a consequence, tumor cell proliferation, reduced apoptosis and angiogenesis occur. In addition, cells lacking functioning PTEN, a lipid phosphatase that reduces cellular PIP3, are associated with a variety of human tumours including breast, prostate, endometrial cancers along with melanomas and glioblastomas. Steck et al., Nat. Genetics, 15, 356-362 (1997).

PDK1 function is critical to downstream signaling that results from activation of cells by growth factors because PKB/Akt, p70S6K, and RSK cannot be activated in cells lacking PDK1. Indeed, disrupting the PDK1 gene in mouse embryonic cells prevents activation of PKB/Akt, p70S6K, and RSK. Williams et al., Current Biology 10, 439-447 (2000). Additionally, in an in vivo model, reducing the expression of PDK1 protects mice from developing tumors under conditions where PIP3 is elevated due to the deletion of PTEN. Bayascas et al., Current Biology 15, 1839-1846 (2005). Thus, while not being bound by any specific theory, inhibiting PDK1 function is expected to mitigate tumor cell proliferation by abrogating cell signaling.

Accordingly, there exists a need in the art for small-molecule inhibitors of PDK1 that are useful for treating cancer and other disorders associated with aberrant PDK1 activity.

### SUMMARY OF THE INVENTION

This invention relates to certain thiazole carboxamide derivatives of Formula (I) and pharmaceutically acceptable salts thereof as inhibitors of 3-phosphoinositide-dependent protein kinase (PDK-1):

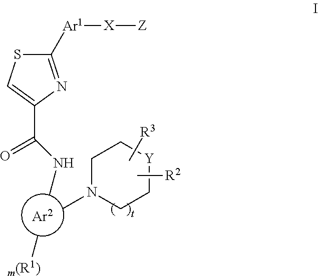

The compounds can be useful in inhibiting the proliferation of cancer cells, and other aberrant conditions where the PDK-1 signaling pathway is overstimulated.

### DETAILED DESCRIPTION OF THE INVENTION

The present invention provides Thiazole Carboximide Compounds and pharmaceutical compositions comprising a Thiazole Carboximide Compound. In addition, the present invention provides methods of using the Thiazole Carboximide Compounds in treating a disease or disorder characterized by excessive or pathologically elevated cell growth, e.g., cancer, in a patient in need of such treatment.

### Compounds

The present invention provides compounds of Formula

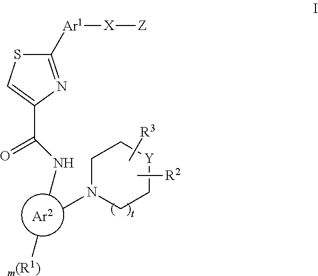

### Wherein

R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆ alkynyl, C₆-C₁₀aryl, C₃-C₈cycloalkyl, 5- to 10-membered heteroaryl, 5- to 10-membered heterocyclyl, 5- to 10-membered heterocyclenyl, C₆-C₁₀arylC₁-C₆alkyl, C₃-C₈cycloalkylalkyl, 5- to 10-membered heteroarylC₁-C₆alkyl, 5- to 10-membered heterocyclylC₁-C₆alkyl and 5- to 10-membered heterocyclenylC₁-C₆alkyl;
R² and R³ are independently selected from H, OH, halo, C₁-C₆ alkyl, (CR^{a}R^{b})_{q}NR^{b}R⁴, (CR^{a}R^{b})_{q}C(O)OR⁴, (CR^{a}R^{b})_{q}OR⁴, (CR^{a}R^{b})_{q}NR^{b}C(O)R^{a}, (CR^{a}R^{b})_{q}NR^{b}C(O)OR^{a}, (CR^{a}R^{b})_{q}NR^{b}C(O)NR^{a}R^{b} and (CR^{a}R^{b})_{q}C(O)NR^{b}R⁴;
or R² and R³ together form a 5 or 6 membered heterocyclic ring with C, O and N atoms, wherein the heterocyclic ring can be optionally substituted with one or more substituents selected from halo, OH, (CR^{a}R^{b})_{q}OR⁴, COOR⁴, O—C₁-C₆ alkyl, NH₂, CN, C₁-C₆ alkyl, C₁-C₆alkenyl, C₁-C₆ alkynyl, halo-C₁-C₆alkyl, O-halo-C₁-C₆alkyl and S-halo-C₁-C₆alkyl;
R^{a} and R^{b} are independently selected from H and C₁-C₆ alkyl;
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})_{q}NR^{a}—, —(CR^{a}R^{b})_{q}O—, —(CR^{a}R^{b})_{q}NR⁴C(O)—, —(CR^{a}R^{b})_{q}NR⁴C(O)NR⁴—, —(CR^{a}R^{b})_{q}NR⁴C(O)O—, —(CR^{a}R^{b})_{q}OC(O)NR⁴—, —(CR^{a}R^{b})_{q}C(O)NR⁴—, —(CR^{a}R^{b})_{q}S(O)₂—, —(CR^{a}R^{b})_{q}SO—, —(CR^{a}R^{b})_{q}S(O)₂NR⁴—, —(CR^{a}R^{b})_{q}S(O)₂NR⁴C(O)—, —(CR^{a}R^{b})_{q}C(O)O—, —(CR^{a}R^{b})_{q}OC(O)—, —(CR^{a}R^{b})_{q}OC(O)O—, and —(CR^{a}R^{b})_{q}S—;

### Y is C or N;

Z is selected from the group consisting of H, C₁-C₆ alkyl, 5- to 10-membered heteroaryl, 5-to 10-membered heterocyclyl, 5- to 10-membered heterocyclenyl, C₆-C₁₀aryl, C₃-C₈cycloalkyl, wherein said alkyl, cycloalkyl, heteroaryl, heterocyclyl, heterocyclenyl or aryl is optionally substituted with one to three substituents selelected from halo, (CR^{a}R^{b})_{q}OR⁴, —O-haloC₁-C₆alkyl, —S-haloC₁-C₆alkyl, (CR^{a}R^{b})_{q}C(O)OR⁴, —(CR^{a}R^{b})_{q}N(R^{a})₂, —(CR^{a}R^{b})_{q}C(O)NHR⁴, —(CR^{a}R^{b})_{q}NR^{a}C(O)R^{a}, CN, C₁-C₆ alkyl, C₁-C₆ alkenyl, C₁-C₆ alkynyl, halo-C₁-C₆alkyl, 5- to 10-membered heteroaryl, 5- to 10-membered heterocyclyl, 5- to 10-membered heterocyclenyl and C₆-C₁₀aryl;
m is independently 0, 1, 2, 3 or 4;
n is independently 1, 2 or 3;
t is independently 0 or 1;
q is independently 0, 1, 2 or 3;
Or a pharmaceutically acceptable salt thereof.

The present invention also provides compounds of Formula IIA:

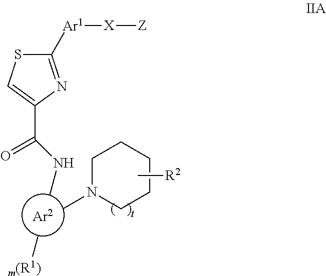

### Wherein

R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆alkynyl and halo-C₁-C₆alkyl;
R² is (CR^{a}R^{b})_{q}NHR⁴ or (CR^{a}R^{b})_{q}C(O)OR⁴;
R^{a} and R^{b} are independently selected from H and C₁-C₆ alkyl;
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})ₙO—, —(CR^{a}R^{b})ₙNR^{a}—, —(CR^{a}R^{b})ₙNR⁴C(O)—, —(CR^{a}R^{b})ₙC(O)NR⁴—, and —(CR^{a}R^{b})S—;
Z is selected from the group consisting of C₁-C₆ alkyl, C₆-C₁₀aryl, 5- to 10-membered heteroaryl and 5- to 10-membered heterocyclyl, wherein said alkyl, heteroaryl, heterocyclyl or aryl is optionally substituted with one to three substituents selelected from halo, (CR^{a}R^{b})_{q}OR⁴, —O-haloC₁-C₆alkyl, —S-haloC₁-C₆alkyl, (CR^{a}R^{b})_{q}C(O)OR⁴, —(CR^{a}R^{b})_{q}N(R^{a})₂, —(CR^{a}R^{b})_{q}C(O)NHR⁴, —(CR^{a}R^{b})_{q}NR^{a}C(O)R^{a}, CN, C₁-C₆ alkyl, C₁-C₆ alkenyl, C₁-C₆ alkynyl, halo-C₁-C₆alkyl, 5- to 10-membered heteroaryl, 5- to 10-membered heterocyclyl, 5- to 10-membered heterocyclenyl and C₆-C₁₀aryl;
in is independently 0, 1, 2, 3 or 4;
n is independently 1, 2 or 3;
t is independently 0 or 1;
q is independently 0, 1, 2 or 3;
Or a pharmaceutically acceptable salt thereof.

The invention also provides compounds of Formula IIB,

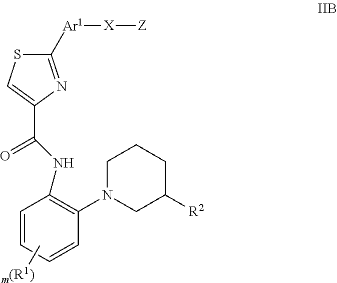

Wherein all other substituents are as defined above.

The invention also provides compounds of Formula IIC,

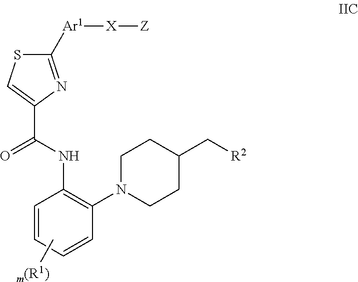

Wherein all other substituents are as defined above.

In one embodiment, R² is NH₂ or —CH₂—NH₂.

The invention also provides compounds of Formula IID,

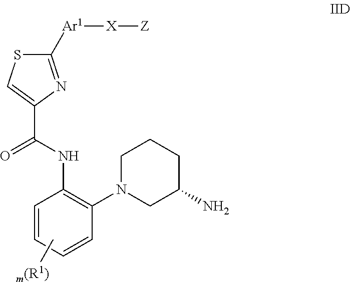

Wherein all other substituents are as defined above.

The invention also provides compounds of Formula IIE:

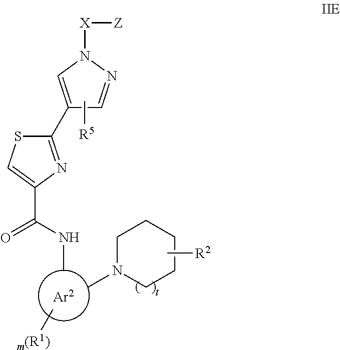

Wherein all other substituents are as defined above.

The invention also provides compounds of Formula IIIA:

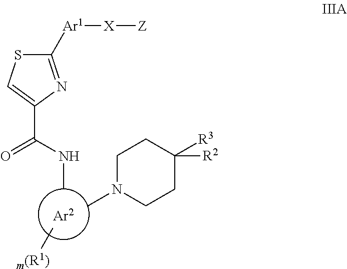

### Wherein

R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆ alkynyl and halo-C₁-C₆alkyl;
R² is (CR^{a}R^{b})_{q}C(O)NHR⁴;
R³ is (CR^{a}R^{b})_{q}NHR⁴;
or R² and R³ together form a 5 or 6 membered heterocyclic ring with C, O and N atoms, wherein the heterocyclic ring can be optionally substituted with one or more substituents selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
R^{a} and R^{b} are independently selected from H and C₁-C₆ alkyl;
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})ₙNR^{a}—, —(CR^{a}R^{b})ₙNR⁴C(O)—, —(CR^{a}R^{b})ₙC(O)NR⁴—, and —(CR^{a}R^{b})ₙS—;
Z is selected from the group consisting of C₁-C₆ alkyl, C₆-C₁₀aryl, 5- to 10-membered heteroaryl and 5- to 10-membered heterocyclyl, wherein said alkyl, heteroaryl, heterocyclyl or aryl is optionally substituted with one to three substituents selelected from halo, —(CR^{a}R^{b})_{q}OR⁴, —O-haloC₁-C₆alkyl, —S-haloC₁-C₆alkyl, (CR^{a}R^{b})_{q}C(O)OR⁴, —(CR^{a}R^{b})_{q}N(R^{a})₂, —(CR^{a}R^{b})_{q}C(O)NHR⁴, —(CR^{a}R^{b})_{q}NR^{a}C(O)R^{a}, CN, C₁-C₆ alkyl, C₁-C₆ alkenyl, C₁-C₆ alkynyl, halo-C₁-C₆alkyl, 5- to 10-membered heteroaryl, 5- to 10-membered heterocyclyl, 5- to 10-membered heterocyclenyl and C₆-C₁₀aryl;
m is independently 0, 1, 2, 3 or 4;
n is independently 1, 2 or 3;
q is independently 0, 1, 2 or 3;
Or a pharmaceutically acceptable salt thereof.

In one embodiment, R² is CONH₂ and R³ is NHMe.

The invention also provides compounds of Formula IIIB:

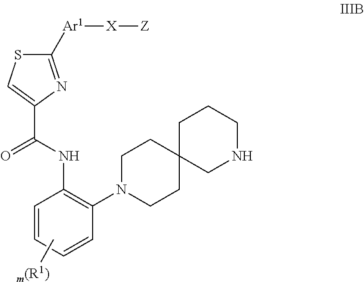

Wherein all other substituents are as defined under Formula III.

The invention also provides compounds of Formula IIIC:

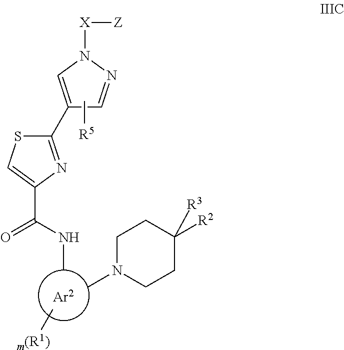

Wherein all other substituents are as defined under Formula III.

In one embodiment, Ar¹ is pyrazolyl, optionally substituted with one to three substituents selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl.

In one embodiment, Ar² is a 6-membered aryl or heteroaryl. In another embodiment, Ar² is phenyl or pyridyl. In another embodiment, Ar² is phenyl.

In another embodiment, —X—Z is —CH₂-pyridyl, —CH₂-phenyl, —CH₂—CH₂-phenyl or CH₂-thienyl, wherein the pyridyl, phenyl or thienyl is optionally substituted with fluoro.

In one embodiment, Y is C.

Specific embodiments depicting non-limiting Examples of the above Formulas are provided in the Experimental Section hereinbelow.

Specific examples of the compounds of the instant invention include:
N-[5-amino-2-(3(R)-amino-1-piperidinyl)phenyl]-2-[1-[3-(dimethylamino)propyl]-1H-pyrazol-4-yl]-4-thiazolecarboxanaide;
N-[5-amino-2-(3(R)-amino-1-piperidinyl)phenyl]-2-[1-(2-thienylmethyl)-1H-pyrazol-4-yl]-4-thiazolecarboxamide;
N-[5-amino-2-(3(R)-amino-1-piperidinyl)phenyl]-2-[1-(phenylmethyl)-1H-pyrazol-4-yl]-4-thiazolecarboxamide;
N-[5-amino-2-(3(R)-amino-1-piperidinyl)phenyl]-2-[1-[2-(4-morpholinyl)ethyl]-1H-pyrazol-4-yl]-4-thiazolecarboxamide;
1-[4-amino-2-[[[2-[1-(2-thienylmethyl)-1H-pyrazol-4-yl]-4-thiazolyl]carbonyl]amino]phenyl]-4-(methylamino)-4-piperidinecarboxamide;
N-[2-[4-(aminomethyl)-1-piperidinyl]-3-fluorophenyl]-2-[1-(2-thienylmethyl)-1H-pyrazol-4-yl]-4-thiazolecarboxamide;
N-[5-amino-2-(2,9-diazaspiro[5.5]undec-9-yl)phenyl]-2-[1-(2-thienylmethyl)-1H-pyrazol-4-yl]-4-thiazolecarboxamide;
N-[5-amino-2-[4-(aminomethyl)-1-piperidinyl]phenyl]-2-[1-(2-thienylmethyl)-1H-pyrazol-4-yl]-4-thiazolecarboxamide;
N-[5-amino-2-(3(R)-amino-1-piperidinyl)phenyl]-2-[1-(4-pyridinylmethyl)-1H-pyrazol-4-yl]-4-thiazolecarboxamide;
N-[5-amino-2-(3(R)-amino-1-pyrrolidinyl)phenyl]-2-[1-(2-thienylmethyl)-1H-pyrazol-4-yl]-4-thiazolecarboxamide;
ethyl 1-[4-amino-2-[[[2-[1-(2-thienylmethyl)-1H-pyrazol-4-yl]-4-thiazolyl]carbonyl]amino]phenyl]-3-piperidineearboxylate;
ethyl 1-[4-amino-2-[[[2-[1-(2-thienylmethyl)-1H-pyrazol-4-yl]-4-thiazolyl]carbonyl]amino]phenyl]-4-piperidinecarboxylate;
N-[5-amino-2-(3(R)-amino-1-piperidinyl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-[(3,4-difluorophenyl)methyl]-1H-pyrazol-4-yl]-4-thiazolecarboxamide;
N-[5-amino-2-(1,4-dioxa-8-azaspiro[4.5]dec-8-yl)phenyl]-2-[1-[(3-fluorophenyl)methyl]-1H-pyrazol-4-yl]-4-thiazolecarboxamide;
N-[5-amino-2-(3(R)-amino-1-piperidinyl)phenyl]-2-[1-[(3,5-difluorophenyl)methyl]-1H-pyrazol-4-yl]-4-thiazolecarboxamide;
N-[5-amino-2-(3(R)-amino-1-piperidinyl)phenyl]-2-[1-(2-phenylethyl)-1H-pyrazol-4-yl]-4-thiazolecarboxamide;
N-[5-amino-2-(3(R)-amino-1-piperidinyl)phenyl]-2-[1-(3-pyridinylmethyl)-1H-pyrazol-4-yl]-4-thiazolecarboxamide;
N-[5-amino-2-(3(R)-amino-1-piperidinyl)phenyl]-2-[1-[(3-fluorophenyl)methyl]-1H-pyrazol-4-yl]-4-thiazolecarboxamide;
N-[5-amino-2-(2,9-diazaspiro[5.5]undec-9-yl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-[(2-fluorophenyl)methyl]-1H-pyrazol-4-yl]-4-thiazolecarboxamide;
N-[5-amino-2-(3(R)-amino-1-piperidinyl)phenyl]-2-[1-[(2,5-difluorophenyl)methyl]-1H-pyrazol-4-yl]-4-thiazolecarboxamide;
N-[2-(3(R)-amino-1-piperidinyl)-3-fluorophenyl]-2-[1-(2-thienylmethyl)-1H-pyrazol-4-yl]-4-thiazolecarboxamide;
N-[5-amino-2-[4-(aminomethyl)-1-piperidinyl]phenyl]-2-[1-(2-pyridinylmethyl)-1H-pyrazol-4-yl]-4-thiazolecarboxamide;
N-[2-(3(R)-amino-1-piperidinyl)-3-fluorophenyl]-2-[1-(phenylmethyl)-1H-pyrazol-4-yl]-4-thiazolecarboxamide
N-[5-amino-2-(3(R)-amino-1-piperidinyl)phenyl]-2-[1-(2-methoxyethyl)-1H-pyrazol-4-yl]-4-thiazolecarboxamide;
N-[2-(3(R)-amino-1-piperidinyl)-3-fluorophenyl]-2-[1-(2-pyridinylmethyl)-1H-pyrazol-4-yl]-4-thiazolecarboxamide;
N-[5-amino-2-(3(R)-amino-1-pyrrolidinyl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-(1-phenylethyl)-1H-pyrazol-4-yl]-4-thiazolecarboxamide;
N-[2-(3(R)-amino-1-piperidinyl)-3-fluorophenyl]-2-[1-(3-pyridinylmethyl)-1H-pyrazol-4-yl]-4-thiazolecarboxamide;
N-[2-(3(R)-amino-1-piperidinyl)-3-fluorophenyl]-2-[1-[(2,6-difluorophenyl)methyl]-1H-pyrazol-4-yl]-4-thiazolecarboxamide;
N-[2-(3(R)-amino-1-piperidinyl)-3-fluorophenyl]-2-[1-[[2-(trifluoromethoxy)phenyl]methyl]-1H-pyrazol-4-yl]-4-thiazolecarboxamide;
N-[5-amino-2-(1,4-dioxa-8-azaspiro[4.5]dec-8-yl)phenyl]-2-[1-(2-thienylmethyl)-1H-pyrazol-4-yl]-4-thiazolecarboxamide;
N-[2-(3(R)-amino-1-piperidinyl)-3-fluorophenyl]-2-[1-[[2-fluoro-6-(trifluoromethyl)phenyl]methyl]-1H-pyrazol-4-yl]-4-thiazolecarboxamide;
N-[2-(3(R)-amino-1-piperidinyl)-3-fluorophenyl]-2-[1-[[2-(difluoromethoxy)phenyl]methyl]-1H-pyrazol-4-yl]-4-thiazolecarboxamide;
N-[2-(3(R)-amino-1-piperidinyl)-3-fluorophenyl]-2-[1-[[2-[(trifluoromethyl)thio]phenyl]methyl]-1H-pyrazol-4-yl]-4-thiazolecarboxamide;
N-[2-(3(R)-amino-1-piperidinyl)-3-fluorophenyl]-2-[1-[(2,4,6-trifluorophenyl)methyl]-1H-pyrazol-4-yl]-4-thiazolecarboxamide;
2-[1-[(2,5-difluorophenyl)methyl]-1H-pyrazol-4-yl]-N-[2-(1-piperazinyl)phenyl]-4-thiazolecarboxamide;
N-[2-(3(R)-amino-1-piperidinyl)-3-fluorophenyl]-2-[1-[2-(phenylmethoxy)ethyl]-1H-pyrazol-4-yl]-4-thiazolecarboxamide;
2-[1-[2-(4-chlorophenoxy)ethyl]-1H-pyrazol-4-yl]-N-[2-(1-piperazinyl)phenyl]-4-thiazolecarboxamide;
N-[2-(3(R)-amino-1-piperidinyl)-3-fluorophenyl]-2-[1-[[2-(hydroxymethyl)phenyl]methyl]-1H-pyrazol-4-yl]-4-thiazolecarboxamide;
N-[2-(3(R)-amino-1-piperidinyl)-3-fluorophenyl]-2-[1-(3-phenoxypropyl)-1H-pyrazol-4-yl]-4-thiazolecarboxamide;
2-[[[2-[4-[4-[[[5-amino-2-(3(R)-amino-1-piperidinyl)phenyl]amino]carbonyl]-2-thiazolyl]-1H-pyrazol-1-yl]ethyl]amino]carbonyl]benzoic acid;
methyl 2-[[4-[4-[[[2-(3(R)-amino-1-piperidinyl)-3-fluorophenyl]amino]carbonyl]-2-thiazolyl]-1H-pyrazol-1-yl]methyl]-3-fluorobenzoate;
N-[2-(3(R)-amino-1-piperidinyl)-3-fluorophenyl]-2-[1-[[2-fluoro-6-(hydroxymethyl)phenyl]methyl)-1H-pyrazol-4-yl]-4-thiazolecarboxamide;
N-[2-(3(R)-amino-1-piperidinyl)-3-fluorophenyl]-2-[1-(4-piperidinylmethyl)-1H-pyrazol-4-yl]-4-thiazolecarboxamide;
N-[2-(1-piperazinyl)phenyl]-2-[1-[2-(4-piperidinyl)ethyl]-1H-pyrazol-4-yl]-4-thiazolecarboxamide;
N-[2-(3(R)-amino-1-piperidinyl)-3-fluorophenyl]-2-[1-[(2-bromophenyl)methyl]-1H-pyrazol-4-yl]-4-thiazolecarboxamide;
N-[2-(3(R)-amino-1-piperidinyl)-3-fluorophenyl]-2-[1-[[2-(1h-pyrazol-4-yl)phenyl]methyl]-1H-pyrazol-4-yl]-4-thiazolecarboxamide;
2-[1-[[2-(aminomethyl)phenyl]methyl]-1H-pyrazol-4-yl]-N-[2-(3(R)-amino-1-piperidinyl)-3-fluorophenyl]-4-thiazolecarboxamide;
N-[2-(3(R)-amino-1-piperidinyl)-3-fluorophenyl]-2-[1-[(2,3-difluorophenyl)methyl]-1H-pyrazol-4-yl]-4-thiazolecarboxamide;
   Or a stereoisomer thereof;
   Or a pharmaceutically acceptable salt thereof;
   Or a pharamceutically acceptable salt of the stereoisomer thereof.

### Chemical Definitions

As used herein, “alkyl” is intended to include both branched and straight-chain saturated aliphatic hydrocarbon groups having the specified number of carbon atoms. For example, C₁-C₁₀ as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylaryl”, “alkylcycloalkyl” and “alkylheterocyclyl” the term “alkyl” refers to the alkyl portion of the moiety and does not describe the number of atoms in the heterocyclyl portion of the moiety. In an embodiment, if the number of carbon atoms is not specified, the “alkyl” of “alkylaryl”, “alkylcycloalkyl” and “alkylheterocyclyl” refers to C₁-C₁₂ alkyl and in a further embodiment, refers to C₁-C₆ alkyl.

The term “cycloalkyl” means a monocyclic saturated or unsaturated aliphatic hydrocarbon group having the specified number of carbon, atoms. The cycloalkyl is optionally bridged (i.e., forming a bicyclic moiety), for example with a methylene, ethylene or propylene bridge. The cycloalkyl may be fused with an aryl group such as phenyl, and it is understood that the cycloalkyl substituent is attached via the cycloalkyl group. For example, “cycloalkyl” includes cyclopropyl, methyl-cyclopropyl, 2,2-dimethyl-cyclobutyl, 2-ethyl-cyclopentyl, cyclohexyl, cyclopentenyl, cyclobutenyl and so on.

In an embodiment, if the number of carbon atoms is not specified, “alkyl” refers to C₁-C₁₂ alkyl and in a further embodiment, “alkyl” refers to C₁-C₆ alkyl. In an embodiment, if the number of carbon atoms is not specified, “cycloalkyl” refers to C₃-C₁₀ cycloalkyl and in a further embodiment, “cycloalkyl” refers to C₃-C₇ cycloalkyl. In an embodiment, examples of “alkyl” include methyl, ethyl, n-propyl, i-propyl, n-butyl, t-butyl and i-butyl.

The term “alkylene” means a hydrocarbon diradical group having the specified number of carbon atoms. For example, “alkylene” includes —CH₂—, —CH₂CH₂— and the like. In an embodiment, if the number of carbon atoms is not specified, “alkylene” refers to C₁-C₁₂ alkylene and in a further embodiment, “alkylene” refers to C₁-C₆ alkylene.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

“Aryl” is intended to mean any stable monocyclic, bicyclic or tricyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In one embodiment, “aryl” is an aromatic ring of 6 to 14 carbons atoms, and includes a carbocyclic aromatic group fused with a 5- or 6-membered cycloalkyl group such as indan. Examples of carbocyclic aromatic groups include, but are not limited to, phenyl, naphthyl, e.g. 1-naphthyl and 2-naphthyl; anthracenyl, e.g. 1-anthracenyl, 2-anthracenyl; phenanthrenyl; fluorenonyl, e.g. 9-fluorenonyl, indanyl and the like.

The term heteroaryl, as used herein, represents a stable monocyclic, bicyclic or tricyclic ring of up to 7 atoms in each ring, wherein at least one ring is aromatic and contains carbon and from 1 to 4 heteroatoms selected from the group consisting of O, N and S. In another embodiment, the term heteroaryl refers to a monocyclic, bicyclic or tricyclic aromatic ring of 5- to 14-ring atoms of carbon and from one to four heteroatoms selected from O, N, or S. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. Additional examples of heteroaryl include, but are not limited to pyridyl, e.g., 2-pyridyl (also referred to as α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like.

In an embodiment, “heteroaryl” may also include a “fused polycyclic aromatic”, which is a heteroaryl fused with one or more other heteroaryl or nonaromatic heterocyclic ring. Examples include, quinolinyl and isoquinolinyl, e.g. 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g. 2-benzofuranyl and 3-benzofuranyl; dibenzofuranyl, e.g. 2,3-dihydrobenzofuranyl; dibenzothiophenyl; benzothienyl, e.g. 2-benzothienyl and 3-benzothienyl; indolyl, e.g. 2-indolyl and 3-indolyl; benzothiazolyl, e.g., 2-benzothiazolyl; benzooxazolyl, e.g., 2-benzooxazolyl; benzimidazolyl, e.g. 2-benzoimidazolyl; isoindolyl, e.g. 1-isoindolyl and 3-isoindolyl; benzotriazolyl; purinyl; thianaphthenyl, pyrazinyland the like.

“Heterocyclyl” means a non-aromatic saturated monocyclic, bicyclic, tricyclic or spirocyclic ring system comprising up to 7 atoms in each ring. Preferably, the heterocyclyl contains 3 to 14, or 5 to 10 ring atoms, in which one or more of the atoms in the ring system is an element other than carbon, for example, nitrogen, oxygen, phosphor or sulfur, alone or in combination. There are no adjacent oxygen and/or sulfur atoms present in the ring system. Preferred heterocyclyls contain about 5 to about 6 ring atoms. The heterocycle may be fused with an aromatic aryl group such as phenyl or heterocyclenyl. The prefix aza, oxa or thia before the heterocyclyl root name means that at least a nitrogen, oxygen or sulfur atom, respectively, is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone:

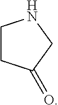

In describing the heteroatoms contained in a specified heterocyclyl group, the expression, “having one to x heteroatoms selected from the group of N, O, P and S” (wherein x is an a specified integer), for example, means that each heteroatom in the specified heterocyclyl is independently selected from the specified selection of heteroatoms. Attachment of a heterocyclyl substituent can occur via a carbon atom or via a heteroatom.

“Heterocyclenyl” means a non-aromatic monocyclic, bicyclic, tricyclic or spirocyclic ring system comprising up to 7 atoms in each ring. Preferably, the heterocyclenyl contains 3 to 14, or 5 to 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen, phosphor or sulfur atom respectively is present as a ring atom. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. An example of such a moiety is pyrrolidinone:

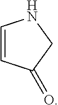

In describing the heteroatoms contained in a specified heterocyclenyl group, the expression, “having one to x heteroatoms selected from the group of N, O, P and S” (wherein x is an a specified integer), for example, means that each heteroatom in the specified heterocyclenyl is independently selected from the specified selection of heteroatoms.

It should also be noted that tautomeric forms such as, for example, the moieties:

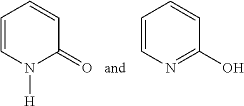 [SMILES: O=c1cccc[nH]1 ; Oc1ccccn1]

are considered equivalent in certain embodiments of this invention.

An “alkylaryl group” is an alkyl group substituted with an aryl group, for example, a phenyl group. Suitable aryl groups are described herein and suitable alkyl groups are described herein. The bond to the parent moiety is through the aryl group.

An “alkylheteroaryl group” is an alkyl group substituted with a heteroaryl group. Suitable heteroaryl groups are described herein and suitable alkyl groups are described herein. The bond to the parent moiety is through the heteroaryl group.

An “alkylheterocyclyl group” is an alkyl group substituted with a heterocyclyl group. Suitable heterocyclyl groups are described herein and suitable alkyl groups are described herein. The bond to the parent moiety is through the heterocyclyl group.

An “alkylheterocyclenyl group” is an alkyl group substituted with a heterocyclenyl group. Suitable heterocyclenyl groups are described herein and suitable alkyl groups are described herein. The bond to the parent moiety is through the heterocyclenyl group.

An “alkylcycloalkyl group” is an alkyl group substituted with a cycloalkyl group. Suitable cycloalkyl groups are described herein and suitable alkyl groups are described herein. The bond to the parent moiety is through the cycloalkyl group.

An “arylalkyl group” is an aryl group substituted with an alkyl group, for example, a phenyl group. Suitable aryl groups are described herein and suitable alkyl groups are described herein. The bond to the parent moiety is through the alkyl group.

A “heteroarylalkyl group” is a heteroaryl group substituted with an alkyl group. Suitable heteroaryl groups are described herein and suitable alkyl groups are described herein. The bond to the parent moiety is through the alkyl group.

A “heterocyclylalkyl group” is a heterocyclyl group substituted with an alkyl group. Suitable heterocyclyl groups are described herein and suitable alkyl groups are described herein. The bond to the parent moiety is through the alkyl group.

A “heterocyclenylalkyl group” is a heterocyclenyl group substituted with an alkyl group. Suitable heterocyclenyl groups are described herein and suitable alkyl groups are described herein. The bond to the parent moiety is through the alkyl group.

A “cycloalkylalkyl group” is a cycloalkyl group substituted with an alkyl group. Suitable cycloalkyl groups are described herein and suitable alkyl groups are described herein. The bond to the parent moiety is through the alkyl group.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

An “alkylamino group” as used herein, is an alkyl group that is attached to a compound via a nitrogen.

As used herein, an “arylalkylamino group” is an arylalkyl group that is attached to a compound via a nitrogen on the alkyl portion of the arylalkyl.

An “alkylsulfonyl group” as used herein, is an alkyl group that is attached to a compound via the sulfur of a sulfonyl group.

When a moiety is referred to as “unsubstituted” or not referred to as “substituted” or “optionally substituted”, it means that the moiety does not have any substituents. When a moiety is referred to as substituted, it denotes that any portion of the moiety that is known to one skilled in the art as being available for substitution can be substituted. The phrase “optionally substituted with one or more substituents” means, in one embodiment, one substituent, two substituents, three substituents, four substituents or five substituents. For example, the substitutable group can be a hydrogen atom that is replaced with a group other than hydrogen (i.e., a substituent group). Multiple substituent groups can be present. When multiple substituents are present, the substituents can be the same or different and substitution can be at any of the substitutable sites. Such means for substitution are well known in the art. For purposes of exemplification, which should not be construed as limiting the scope of this invention, some examples of groups that are substituents are: alkyl, alkenyl or alkynyl groups (which can also be substituted, with one or more substituents), alkoxy groups (which can be substituted), a halogen or halo group (F, Cl, Br, I), hydroxy, nitro, oxo, —CN, —COH, —COOH, amino, azido, N-alkylamino or N,N-dialkylamino (in which the alkyl groups can also be substituted), N-arylamino or N,N-diarylamino (in which the aryl groups can also be substituted), esters (—C(O)—OR, where R can be a group such as alkyl, aryl, etc., which can be substituted), ureas (—NHC(O)—NHR, where R can be a group such as alkyl, aryl, etc., which can be substituted), carbamates (—NHC(O)—OR, where R can be a group such as alkyl, aryl, etc., which can be substituted), sulfonamides (—NHS(O)₂R, where R can be a group such as alkyl, aryl, etc., which can be substituted), alkylsulfonyl (which can be substituted), aryl (which can be substituted), cycloalkyl (which can be substituted) alkylaryl (which can be substituted), alkylheterocyclyl (which can be substituted), alkylcycloalkyl (which can be substituted), and aryloxy.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. For instance those compounds labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Additionally, isotopic substitution of a compound at a site where epimerization occurs may slow or reduce the epimerization process and thereby retain the more active or efficacious form of the compound for a longer period of time.

It is also possible that the compounds of Formula (I) may exist in different tautomeric forms, and all such forms are embraced within the scope of the invention. Also, for example, all keto-enol and imine-enamine forms of the compounds are included in the invention.

### Stereochemistry

When structures of the same constitution differ in respect to the spatial arrangement of certain atoms or groups, they are stereoisomers, and the considerations that are significant in analyzing their interrelationships are topological. If the relationship between two stereoisomers is that of an object and its nonsuperimposable mirror image, the two structures are enantiomeric, and each structure is said to be chiral. Stereoisomers also include diastereomers, cis-trans isomers and conformational isomers. Diastereoisomers can be chiral or achiral, and are not mirror images of one another. Cis-trans isomers differ only in the positions of atoms relative to a specified planes in cases where these atoms are, or are considered as if they were, parts of a rigid structure. Conformational isomers are isomers that can be interconverted by rotations about formally single bonds. Examples of such conformational isomers include cyclohexane conformations with chair and boat conformers, carbohydrates, linear alkane conformations with staggered, eclipsed and gauche confomers, etc. See J. Org. Chem. 35, 2849 (1970)

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, enantiomers are identical except that they are non-superimposable mirror images of one another. A mixture of enantiomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the Formulas of the invention, it is understood that both the (R) and (S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the compounds of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

This invention is also intended to encompass pro-drugs of the Thiazole Carboximide compounds disclosed herein. A prodrug of any of the compounds can be made using well-known pharmacological techniques.

### Pharmaceutically Acceptable Salts

The Thiazole Carboximide compounds described herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts organic and inorganic acids, for example, acid addition salts which may, for example, be hydrochloric acid, sulphuric acid, methanesulphonic acid, fumaric acid, maleic acid, succinic acid, acetic acid, benzoic acid, oxalic acid, citric acid, tartaric acid, carbonic acid, phosphoric acid, trifluoroacetic acid, formic acid and the like. Pharmaceutically acceptable salts can also be prepared from by treatment with inorganic bases, for example, sodium, potassium, ammonium, calcium, or ferric hydroxides, and such organic bases as isopropylamine, trimethylamine, 2-ethylamino ethanol, histidine, procaine, and the like. Pharmaceutically acceptable salts can also be formed from elemental anions such as chlorine, bromine and iodine.

The active compounds disclosed can, as noted above, also be prepared in the form of their hydrates. The term “hydrate” includes but is not limited to hemihydrate, monohydrate, dihydrate, trihydrate, tetrahydrate and the like.

The active compounds disclosed can, as noted above, also be prepared in the form of a solvate with any organic or inorganic solvent, for example alcohols such as methanol, ethanol, propanol and isopropanol, ketones such as acetone, aromatic solvents and the like.

The active compounds disclosed can also be prepared in any solid or liquid physical form. For example, the compound can be in a crystalline form, in amorphous form, and have any particle size. Furthermore, the compound particles may be micronized, or may be agglomerated, particulate granules, powders, oils, oily suspensions or any other form of solid or liquid physical form.

The compounds of the present invention may also exhibit polymorphism. This invention further includes different polymorphs of the compounds of the present invention. The term “polymorph” refers to a particular crystalline state of a substance, having particular physical properties such as X-ray diffraction, IR spectra, melting point, and the like.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

### Methods of Treatment

The Thiazole Carboximide Compounds can be useful in human and veterinary medicine in the therapy of proliferative diseases such as cancer other non-cancer proliferative disorders. The Thiazole Carboximide Compounds are useful where inhibiting PDK1 or inhibiting PDK1 variants is indicated, such as in treating various diseases associated with abnormal PDK1 signaling and/or abnormal signaling upstream or downstream of PDK1 (or variants thereof), such as that related to up-regulated activity of one or more receptor tyrosine kinases, Ras, PDK1, PKB/Akt, RSK, PKC, 70S6K, or SGK. In some embodiments, the compounds of the invention are useful in inhibiting PDK1 variants wherein the wild type PDK1 contains one or more point mutations, insertions, or deletions. Examples of PDK1 variants include as PDK1T354M and PDK1D527E.

While not being bound by any specific theory, it is believed that the Thiazole Carboximide Compounds are useful in treating proliferative diseases such as cancer and other proliferative diseases because of their PDK1 inhibitory activity.

The general value of the compounds of the invention in inhibiting PDK1 can be determined, for example, using the fluorescence polarization-based assay described in Example 3. In addition, the general value of the compounds of the invention in inhibiting PDK1 function can be evaluated using other known assays such as those described in Xu et al. in 3. Biomol. Screen. 14, 1257-1262 (2009).

The Thiazole Carboximide Compounds can be used to treat diseases and disorders characterized by excessive or pathologically elevated cell growth such as is characteristic of various cancers and non-cancer proliferative disorders. Examples of cancers for which the Thiazole Carboximide Compounds can be useful, include lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer, uterine corpus cancer, uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, and pharyngeal cancer, laryngeal cancer, small intestinal cancer, non-Hodgkin's lymphoma, and villous colon adenoma.

In some embodiments, the compounds of the invention are used to treat cancers of the prostate, lung, colon, or breast.

Examples of non-cancer proliferative disorders for which the Thiazole Carboximide Compounds can be useful include neuro-fibromatosis, atherosclerosis, pulmonary fibrosis, arthritis, psoriasis, glomerulonephritis, restenosis, proliferative diabetic retinopathy (PDR), hypertrophic scar formation, inflammatory bowel disease, transplantation rejection, angiogenesis, and endotoxic shock.

Thus, in one embodiment the invention provides a method of treating a patient (e.g., human) having a disease or disorder characterized by excessive or pathologically elevated cell growth by administering a therapeutically effective amount of a Thiazole Carboximide Compound, or a pharmaceutically acceptable salt of said compound to the patient. In some embodiments, the disease or disorder being treated is a cancer. In other embodiments, the disease or disorder being treated are non-cancer proliferative disorders.

The present invention provides a method of treating cancer comprising the step of administering to a subject a therapeutically effective amount of the Thiazole Carboximide Compounds. The present invention also provides the Use of the Thiazole Carboximide Compounds for the preparation of a medicament for the treatment of cancer. The invention also provides the Thiazole Carboximide Compounds for use in the treatment of cancer.

### DEFINITIONS

As used herein, the term “therapeutically effective amount” means that amount of active compound or pharmaceutical agent that elicits the biological or medicinal response in a tissue, system, animal or human that is being sought by a researcher, veterinarian, medical doctor or other clinician. The therapeutic effect is dependent upon the disease or disorder being treated or the biological effect desired. As such, the therapeutic effect can be a decrease in the severity of symptoms associated with the disease or disorder and/or inhibition (partial or complete) of progression of the disease. The amount needed to elicit the therapeutic response can be determined based on the age, health, size and sex of the subject. Optimal amounts can also be determined based on monitoring of the subject's response to treatment.

Further, a therapeutically effective amount, can be an amount that selectively induces terminal differentiation, cell growth arrest and/or apoptosis of neoplastic cells, or an amount that induces terminal differentiation of tumor cells.

The method of the present invention is intended for the treatment or chemoprevention of human patients with cancer. However, it is also likely that the method would be effective in the treatment of cancer in other subjects. “Subject”, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

### Combination Therapy

The compounds of the present invention can be administered alone or in combination with other therapies suitable for the disease or disorder being treated. Where separate dosage formulations are used, the compound and the other therapeutic agent can be administered at essentially the same time (concurrently) or at separately staggered times (sequentially). The pharmaceutical combination is understood to include all these regimens. Administration in these various ways are suitable for the present invention as long as the beneficial therapeutic effect of the compound and the other therapeutic agent are realized by the patient at substantially the same time. In an embodiment, such beneficial effect is achieved when the target blood level concentrations of each active drug are maintained at substantially the same time.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Therefore, the present invention encompasses pharmaceutical compositions comprising a therapeutically effective amount of the compound of the invention and a pharmaceutically acceptable carrier and optionally other threrapeutic ingredients, such as an anti-cancer agent. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents, agents that interfere with cell cycle checkpoints, agents that interfere with receptor tyrosine kinases (RTKs) and cancer vaccines. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, diethylstibestral, tamoxifen, raloxifene, idoxifene, LY353381, LY117081, toremifene, fluoxymestero, 1fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

Other hormonal agents include: aromatase inhibitors (e.g., aminoglutethimide, anastrozole and tetrazole), luteinizing hormone release hormone (LHRH) analogues, ketoconazole, goserelin acetate, leuprolide, megestrol acetate and mifepristone.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, chlorambucil, cyclophosphamide, ifosfamide, mechlorethamine, melphalan, uracil mustard, thiotepa, busulfan, carmustine, lomustine, streptozocin, tasonermin, lonidamine, carboplatin, altretamine, dacarbazine, procarbazine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, doxorubicin, daunorubicin, idarubicin, anthracenedione, bleomycin, mitomycin C, dactinomycin, plicatomycin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include vincristine, vinblastine, vindesine, vinzolidine, vinorelbine, vindesine sulfate, 3′,4′-didehydro-4′-deoxy-8′-norvincaleukoblastine, podophyllotoxins (e.g., etoposide (VP-16) and teniposide (VM-26)), paclitaxel, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)carnptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678, WO 03/39460 and WO2003/079973, WO2003/099211, WO2004/039774, WO2003/105855, WO2003/106417. In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Examples of “histone deacetylase inhibitors” include, but are not limited to, SAHA, TSA, oxamflatin, PXD101, MG98, valproic acid and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, floxuridine, methotrexate, leucovarin, hydroxyurea, thioguanine (6-TG), mercaptopurine (6-MP), cytarabine, pentostatin, fludarabine phosphate, eladribine (2-CDA), asparaginase, gemcitabine, alano sine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabin furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epideunal-derived, fibroblast-derived, or platelet derived growth factors, MMP (matrix metalloprotease) inhibitors, integrin blockers, interferon-α, interleukin-12, erythropoietin (epoietin-α), granulocyte-CSF (filgrastin), granulocyte, macrophage-CSF (sargramostim), pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs shown as described by Bume-Jensen and Hunter, Nature, 411:355-365, 2001.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of CD20 (rituximab), inhibitors of cytokine receptors, inhibitors of MET, inhibitors of PI3K family kinase (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573). Such agents include small molecule inhibitor compounds and antibody antagonists.

Examples of mTOR inhibitors include ridaforolimus, temsirolimus, everolimus, a rapamycin-analog. Ridaforolimus, also known as AP 23573, MK-8669 and deforolimus, is a unique, non-prodrug analog of rapmycin that has antiproliferative activity in a broad range of human tumor cell lines in vitro and in murine tumor xenograft models utilizing human tumor cell lines. Ridaforolimus has been administered to patients with advanced cancer and is currently in clinical development for various advanced malignancies, including studies in patients with advanced soft tissue or bone sarcomas. Thus far, these trials have demonstrated that ridaforolimus is generally well-tolerated with a predictable and manageable adverse even profile, and possess anti-tumor activity in a broad range of cancers. A description and preparation of ridaforolimus is described in U.S. Pat. No. 7,091,213 to Ariad Gene Therapeutics, Inc.

Temsirolirnus, also known as Torisel®, is currently marketed for the treatment of renal cell carcinoma. A description and preparation of temsirolimus is described in U.S. Pat. No. 5,362,718 to American Home Products Corporation. Everolimus, also known as Certican® or RAD001, marketed by Novartis, has greater stability and enhanced solubility in organic solvents, as well as more favorable pharmokinetics with fewer side effects than rapamycin (sirolimus). Everolimus has been used in conjunction with microemulsion cyclosporin (Neoral®, Novartis) to increase the efficacy of the immunosuppressive regime.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, imatinib (STI571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-8 are the nuclear peroxisome proliferator-activated receptors γ and δ. The expression of PPAR-γ on endothelial cells and its involvement in angiogenesis has been reported in the literature (see J Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), Duc-4, NF-1, NF-2, RB, WT1, BRCA1, BRCA2, a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, bacillus Calmette-Guerin, octreotide, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to anastrozole, letrozole and exemestane.

A compound of the instant invention may also be useful for treating or preventing cancer in combination with siRNA therapeutics.

A compound of the instant invention may also be useful for treating or preventing cancer in combination withcompounds which induce terminal differentiation of the neoplastic cells. Suitable differentiation agents include the compounds disclosed in any one or more of the following references.
a) Polar compounds (Marks et al (1987); Friend, C., Scher, W., Holland, J. W., and Sato, T. (1971) Proc. Natl. Acad. Sci. (USA) 68: 378-382; Tanaka, M., Levy, J., Terada, M., Breslow, R., Rifkind, R. A., and Marks, P. A. (1975) Proc. Natl. Acad. Sci. (USA) 72: 1003-1006; Reuben, R. C., Wife, R. L., Breslow, R., Rifkind, R. A., and Marks, P. A. (1976) Proc. Natl. Acad. Sci. (USA) 73: 862-866);
b) Derivatives of vitamin D and retinoic acid (Abe, E., Miyaura, C., Sakagami, H., Takeda, M., Konno, K., Yamazaki, T., Yoshika, S., and Suda, T. (1981) Proc. Natl. Acad. Sci. (USA) 78: 4990-4994; Schwartz, E. L., Snoddy, J. R., Kreutter, D., Rasmussen, H., and Sartorelli, A. C. (1983) Proc. Am. Assoc. Cancer Res. 24: 18; Tanenaga, K., Hozumi, M., and Sakagami, Y. (1980) Cancer Res. 40: 914-919);
c) Steroid hormones (Lotem, J. and Sachs, L. (1975) Int. J. Cancer 15: 731-740);
d) Growth factors (Sachs, L. (1978) Nature (Lond.) 274: 535, Metcalf, D. (1985) Science, 229: 16-22);
e) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354);
f) Tumor promoters (Huberman, E. and Callaham, M. F. (1979) Proc. Natl. Acad. Sci. (USA) 76: 1293-1297; Lottem, J. and Sachs, L. (1979) Proc. Natl. Acad. Sci. (USA) 76: 5158-5162); and
g) inhibitors of DNA or RNA synthesis (Schwartz, E. L. and Sartorelli, A. C. (1982) Cancer Res. 42: 2651-2655, Terada, M., Epner, E., Nudel, U., Salmon, J., Fibach, E., Rifkind, R. A., and Marks, P. A. (1978) Proc. Natl. Acad. Sci. (USA) 75: 2795-2799; Morin, M. J. and Sartorelli, A. C. (1984) Cancer Res 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238).

A compound of the instant invention may also be useful for treating or preventing cancer in combination with γ-secretase inhibitors.

Also included in the scope of the claims is a method of treating cancer that comprises administering a therapeutically effective amount of a compound of Formula I in combination with radiation therapy and/or in combination with a second compound selected from: an estrogen receptor modulator, an androgen receptor modulator, a retinoid receptor modulator, a cytotoxiccytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The compounds of the instant invention are useful in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bendamustine hydrochloride (Treanda); bevacuzimab (Avastin®); bexarotene capsules (Targretin®); bexarotene gel (Targretin); bleomycin (Blenoxane®); bortezomib (Velcade®); busulfan intravenous (Busulfex); busulfan oral (Myleran®); calusterone (Methosarb®); capecitabine (Xeloda®), carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); dalteparin sodium injection (Fragmin®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); etoposide phosphate (Etopophos®); etoposide, VP-16 (Vepeside); everolimus tablets (Afinitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); iobenguane I 123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); Rasburicase (Elitek®); raloxifene hydrochloride (Evista®); Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); vairubicin (Valstar®); vinblastine (Velban®); vincristine (Oncovin®); vinorelbine (Navelbine®); vorinostat (Zolinza®); and zoledronate (Zometa®).

Non-limiting examples of other suitable anti-cancer agents for combination with the instant compounds are selected from the group consisting of a Cytostatic agent, Cisplatin, Deforolimus (described in PCT publication No. 2003/064383), Doxorubicin, liposomal doxorubicin (e.g., Caelyx®, Myocet®, Doxil®), Taxotere, Taxol, Etoposide, Irinotecan, Camptostar, Topotecan, Paclitaxel, Docetaxel, Epothilones, Tamoxifen, 5-Fluorouracil, Methoxtrexate, Temozolomide, cyclophosphamide, SCH 66336, R115777®, L778,123®, BMS 214662®, Iressa®, Tarceva®, Antibodies to EGFR, antibodies to IGFR (including, for example, those published in US 2005/0136063 published Jun. 23, 2005), ESK inhibitors, KSP inhibitors (such as, for example, those published in WO 2006/098962 and WO 2006/098961; ispinesib, SB-743921 from Cytokinetics), Centrosome associated protein E (“CENP-E”) inhibitors (e.g., GSK-923295), Gleevec®, Intron, Ara-C, Adriamycin, Cytoxan, Gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6 Mercaptopurine, 6 Thioguanine, Fludarabine phosphate, Oxaliplatin, Leucovirin, ELOXATIN™, Vinblastine, Vincristine, Vindesine, Bleomycin, Dactinomycin, Daunorubicin, Doxorubicin, Epirubicin, Idarubicin, Mithramycin, Deoxycoformycin, Mitomycin C, L Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, bortezomib (“Velcade”), Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225®, Satriplatin, mylotarg, Avastin, Rituxan, Panitubimab, Sutent, Sorafinib, Sprycel (dastinib), Nilotinib, Tykerb (Lapatinib) and Campath.

In one embodiment, the invention provides a method of treating cancer, the method comprising administering an amount of a Thiazole Carboximide Compound or a pharmaceutically acceptable salt thereof, and an amount of one additional anticancer agent selected from the group consisting of Adriamycin, Altretamine, Amidox, Aminoglutethimide, Amsacrine, Anastrazole, Antibodies to EGFR, 3-AP, Aphidicolon, Ara-C, Arsenic trioxide, L Asparaginase, Bevacizumab, Bleomycin, BMS 214662, Bortezomib, Busulfan, Campath, Camptostar, Capecitabine, Carboplatin, Carmustine, Centrosome associated protein E (“CENP-E”) inhibitors, Cetuximab, Cladribine, Chlorambucil, Chlormethine, Chlorotrianisene, Cisplatin, Clofarabine, cyclophosphamide, Cytarabine, a Cytostatic agent, Cytoxan, Dacarbazine, Dactinomycin, Daunorubicin, Dasatinib, Deforolimus, Deoxycoformycin, Didox, Diethylstilbestrol, Docetaxel, Doxorubicin, Dromostanolone, Droloxafine, Epirubicin, Epothilones, ERK inhibitors, Erlotinib, Etoposide, 17α-Ethinylestradiol, Estramustine, Exemestane, Floxuridine, Fludarabine, Fludarabine phosphate, 5-Fluorouracil, Fluoxymesterone, Flutamide, Fulvestrant, Gefitinib, Gemcitabine, Gemtuzumab ozogamcicin, Goserelin, GSK-923295, Hexamethylmelamine, Hydroxyprogesterone, Hydroxyurea, Ibritumomab Tiuxetan, Idarubicin, Ifosfamide, Imatinib mesylate, Intron, Irinotecan, ispinesib, KSP inhibitors, L778,123, Lapatinib, Leucovirin, Leuprolide, Lerozole, Letrazole, Levamisole, Liposomal Doxorubicin, Liposomal, Lomustine, Lonafamib, Medroxyprogesteroneacetate, Megestrolacetate, Melphalan, 6 Mercaptopurine, Methoxtrexate, Methylprednisolone, Methyltestosterone, Mithramycin, Mitomycin C, Mitotane, Mitoxantrone, Navelbene, Nilotinib, Oxaliplatin, Paclitaxel, Panitubimab, Pentostatin, Pipobroman, Porfimer, Prednisolone, Prednisone propionate, Procarbazine, Reloxafine, Rituximab, Satriplatin, SB-743921, Sml1, Sorafinib, Streptozocin, Sunitinib, Tamoxifen, Taxotere, Taxol, Temozolomide, Teniposide, Testolactone, Testosterone, Tezacitabine, 6 Thioguanine, Thiotepa, Tipifarnib, Topotecan, Toremifene, Tositumomab, Trastuzumab, Triamcinolone, Triapine, Triethylenemelamine, Triethylenethiophosphoramine, Trimidox, Uracil mustard, Vinblastine, Vincristine, Vindesine, and Vinorelbine.

In one embodiment, the invention provides a method of treating cancer, the method comprising administering an amount of a Thiazole Carboximide Compound or a pharmaceutically acceptable salt thereof, and an amount of one or more of a MAP Kinase pathway inhibitor such as bRaf, MEK, or ERK inhibitors to a patient in need thereof.

In another embodiment, the invention provides a method of treating cancer, the method comprising administering an amount of a Thiazole Carboximide Compound or a pharmaceutically acceptable salt thereof, and an amount of one or more of ERK inhibitors (for example, compounds described in WO2008/156739, WO2007/070398, WO 2008/156739 and US publication 2007/0232610) to a patient in need thereof.

In one embodiment, the invention provides a method of treating cancer, the method comprising administering an amount of a Thiazole Carboximide Compound or a pharmaceutically acceptable salt thereof, and an amount of one or more of an anti-IGF-1R antibody. Specific anti-IGF-1R antibodies include, but are not limited to, dalotuzumab, figitumumab, cixutumumab, SHC 717454, Roche R1507, EM164 or Amgen AMG479.

The instant invention also includes a pharmaceutical composition useful for treating or preventing cancer that comprises a therapeutically effective amount of a compound of Formula I and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The use of all of these approaches in combination with the instant compounds described herein are within the scope of the present invention.

### Compositions and Administration

This invention is also directed to pharmaceutical compositions which comprise at least one Thiazole Carboximide Compound, or a pharmaceutically acceptable salt of said compound and at least one pharmaceutically acceptable carrier.

When administered to a patient, the Thiazole Carboximide Compounds can be administered as a component of a composition that comprises a pharmaceutically acceptable carrier or vehicle. The present invention provides pharmaceutical compositions comprising an effective amount of at least one Thiazole Carboximide Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The Thiazole Carboximide Compounds of the present invention may also be delivered transdermally. The transdermal compositions can take the form of creams, lotions, aerosols and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize therapeutic effects, i.e., anti-cancer activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the Thiazole Carboximide Compound is administered orally.

In another embodiment, the Thiazole Carboximide Compound is administered intravenously.

In another embodiment, the Thiazole Carboximide Compound is administered topically.

In still another embodiment, the Thiazole Carboximide Compounds is administered sublingually.

In one embodiment, a pharmaceutical preparation comprising at least one Thiazole Carboximide Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Thiazole Carboximide Compound(s) by weight or volume. In various embodiments, the present compositions can contain, in one embodiment, from about 1% to about 70% or from about 5% to about 60% of the Thiazole Carboximide Compound(s) by weight or volume.

The quantity of Thiazole Carboximide Compound in a unit dose of preparation may be varied or adjusted from about 0.1 mg to about 5000 mg. In various embodiments, the quantity is from about 10 mg to about 5000 mg, about 10 mg to about 1000 mg, 1 mg to about 500 mg, 1 mg to about 100 mg, and 1 mg to about 50 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

For administration to human patients, the amount and frequency of administration of the Thiazole Carboximide Compounds will be regulated according to the judgment of the attending clinician considering such factors as age, condition and size of the patient as well as severity of the symptoms being treated. Generally, a total daily dosage of the Thiazole Carboximide Compounds range from about 0.1 to about 5000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 5000 mg/day, administered in a single dose or in 2-4 divided doses. In another embodiment, the dosage is from about 100 to about 5000 mg/day, administered in a single dose or in 2-4 divided doses. In still another embodiment, the dosage is from about 500 to about 5000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Thiazole Carboximide Compound or a pharmaceutically acceptable salt thereof; (ii) one or more additional therapeutic agents that are not a Thiazole Carboximide Compound; and (iii) a pharmaceutically acceptable carrier, wherein the amounts in the composition are together effective to treat disease or disorder associated with dysregulated PDK-1 activity, such as a cancer.

### In Vitro and In Vivo METHODS:

The present invention also provides methods of using the Thiazole Carboximide compounds of the present invention for inducing terminal differentiation, cell growth arrest and/or apoptosis of neoplastic cells thereby inhibiting the proliferation of such cells. The methods can be practiced in vivo or in vitro.

In one embodiment, the present invention provides in vitro methods for selectively inducing terminal differentiation, cell growth arrest and/or apoptosis of neoplastic cells, thereby inhibiting proliferation of such cells, by contacting the cells with an effective amount of any one or more of the Thiazole Carboximide compounds described herein.

In a particular embodiment, the present invention relates to an in vitro method of selectively inducing terminal differentiation of neoplastic cells and thereby inhibiting proliferation of such cells. The method comprises contacting the cells under suitable conditions with an effective amount of one or more of the Thiazole Carboximide compounds described herein.

In another embodiment, the invention relates to an in vitro method of selectively inducing cell growth arrest of neoplastic cells and thereby inhibiting proliferation of such cells. The method comprises contacting the cells under suitable conditions with an effective amount of one or more of the Thiazole Carboximide compounds described herein.

In another embodiment, the invention relates to an in vitro method of selectively inducing apoptosis of neoplastic cells and thereby inhibiting proliferation of such cells. The method comprises contacting the cells under suitable conditions with an effective amount of one or more of the Thiazole Carboximide compounds described herein.

In another embodiment, the invention relates to an in vitro method of inducing terminal differentiation of tumor cells in a tumor comprising contacting the cells with an effective amount of any one or more of the Thiazole Carboximide compounds described herein.

Although the methods of the present invention can be practiced in vitro, it is contemplated that the preferred embodiment for the methods of selectively inducing terminal differentiation, cell growth arrest and/or apoptosis of neoplastic cells, and of inhibiting PDK-1 will comprise contacting the cells in vivo, i.e., by administering the compounds to a subject harboring neoplastic cells or tumor cells in need of treatment.

Thus, the present invention provides in vivo methods for selectively inducing terminal differentiation, cell growth arrest and/or apoptosis of neoplastic cells in a subject, thereby inhibiting proliferation of such cells in the subject, by administering to the subject an effective amount of any one or more of the Thiazole Carboximide compounds described herein.

In a particular embodiment, the present invention relates to a method of selectively inducing terminal differentiation of neoplastic cells and thereby inhibiting proliferation of such cells in a subject. The method comprises administering to the subject an effective amount of one or more of the Thiazole Carboximide compounds described herein.

In another embodiment, the invention relates to a method of selectively inducing cell growth arrest of neoplastic cells and thereby inhibiting proliferation of such cells in a subject. The method comprises administering to the subject an effective amount of one or more of the Thiazole Carboximide compounds described herein.

In another embodiment, the invention relates to a method of selectively inducing apoptosis of neoplastic cells and thereby inhibiting proliferation of such cells in a subject. The method comprises administering to the subject an effective amount of one or more of the Thiazole Carboximide compounds described herein.

In another embodiment, the invention relates to a method of treating a patient having a tumor characterized by proliferation of neoplastic cells. The method comprises administering to the patient one or more of the Thiazole Carboximide compounds described herein. The amount of compound is effective to selectively induce terminal differentiation, induce cell growth arrest and/or induce apoptosis of such neoplastic cells and thereby inhibit their proliferation.

### Kits

Another aspect of this invention is a kit comprising a therapeutically effective amount of at least one Thiazole Carboximide Compound, or a pharmaceutically acceptable salt of said compound, and a pharmaceutically acceptable carrier, vehicle or diluent.

Yet another aspect of this invention is a kit comprising an amount of at least one

Thiazole Carboximide Compound, or a pharmaceutically acceptable salt of said compound and an amount of at least one additional anti-cancer agent listed above, wherein the amounts of the two or more active ingredients result in a desired therapeutic effect. In one embodiment, the at least one Thiazole Carboximide Compound and the at least one additional anti-cancer agent are provided in the same container. In one embodiment, the at least one Thiazole Carboximide Compound and the at least one additional anti-cancer agent are provided in separate containers.

The invention is illustrated in the examples in the Experimental Details Section that follows. This section is set forth to aid in an understanding of the invention but is not intended to, and should not be construed to limit in any way the invention as set forth in the claims which follow thereafter.

### EXPERIMENTAL DETAILS SECTION

The following solvents, reagents and reaction conditions may be referred to by their abbreviations:

Aq: aqueous
g or gm: grams
psi: pounds per square inch
pH: concentration of hydronium ions in a solution
° C.: degrees Celsius
h: hours

### THF: Tetrahydrofuran

Et₂O: diethyl ether
SEM: 2-(trimethylsilyl)ethoxymethyl
LC-MS: Liquid chromatography mass spectrometry
DCM: dichloromethane

### N: Normal

ml: milliliter

### NBS: N-Bromosuccinimide

### NCS: N-Chlorosuccinimide

### NIS: N-iodosuccinimide

r.t.: room temperature
MeOH: methanol
DIEA: diisopropylethylamine
EtOAc: ethyl acetate
EtOH: ethanol
DMF: dimethylformamide
wt %: weight percent
m/z: mass per charge
LiOH: lithium hydroxide
DMSO: dimethylsulfoxide
HPLC: high performance liquid chromatography
IPA: isopropanol
Ret: retention
Rt: retention time
RP: reverse phase
ACN: acetonitrile
CH₃CN: acetonitrile
MeCN: acetonitrile
MeI: iodomethane
r.t.: room temperature
pTSA: para-toluene sulfonic acid
CDI: N,N′-carbonyldiimidazole
mg: milligram
PMA: phosphomolybdic acid
LiHMDS: Lithium bis(trimethylsilyl)amide
HMDS: hexamethyldisilazane
Pd/C: palladium on carbon
H₂: hydrogen gas
PdCl₂(dppf): [1,1′-bis(diphenylphosphino)ferrocene]dichloropalladium(II)
μmol: micromole
TFA: trifluoroacetic acid
NMP: N-methyl-2-pyrrolidone
min: minute
DME: dimethylethane
AcOH: acetic acid
BBN: 9-borabicyclo[3.3.1]nonane
BOC: tertiary-butyloxycarbonyl

### M: Molar

mmol: millimolar
DIEA: diisopropylethylamine
Bu₃SnCN: tributyltin cyanide
Pd[P(t-Bu)₃]₂: bis(tributyl)Phosphine) palladium
Pd(PPh₃)₄: tetrakis(triphenylphosphine) palladium
EDCI: 1-ethyl-3-(3-dimethylaminopropyl)-carbodiimide
UV: ultraviolet
LDA: lithium diisopropylamide
Tf: trifluoromethanesulfonyl

### Experimental Section

The compounds of the present invention were prepared by the general methods outlined in the synthetic scheme 1 below.

Scheme 1. A General Synthetic Scheme for the Preparation of Compounds 1 to 52

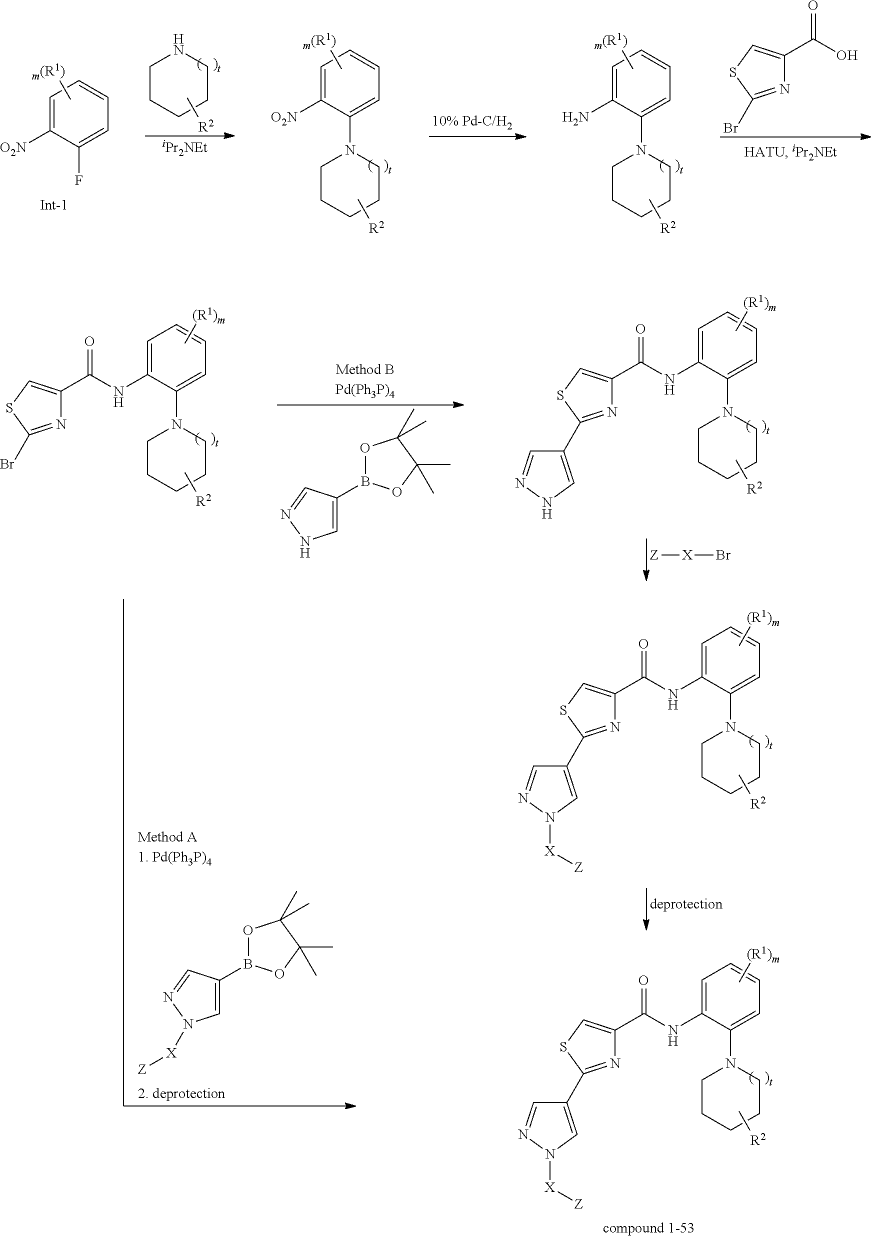

### Example 1

### Procedures for the Preparation of Intermediates Bromothiazoles 8b to 8j (Table 1)

### Preparation of tert-butyl (4-fluoro-3-nitrophenyl)carbamate (Int-2)

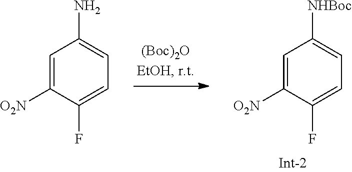 [SMILES: Nc1ccc(F)c([N+](=O)[O-])c1 ; CC(C)(C)OC(=O)Nc1ccc(F)c([N+](=O)[O-])c1]

A mixture of 4-fluoro-3-nitroaniline (30 g, 0.19 mol) and di-tert-butyl dicarbonate (84 g, 0.38 mol) were stirred in ethanol (300 ml) for 7 days. Solvents were removed in vacuum. Chromatographic purification (ethyl acetate-hexane) gave tert-butyl (4-fluoro-3-nitrophenyl)carbamate Int-2 as white solid.

### Preparation of Int-3

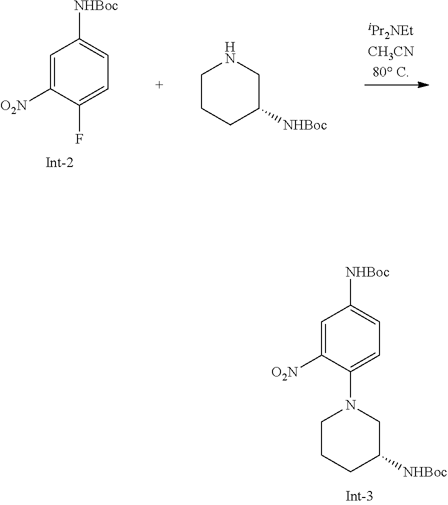 [SMILES: CC(C)(C)OC(=O)Nc1ccc(F)c([N+](=O)[O-])c1 ; CC(C)(C)OC(=O)N[C@@H]1CCCNC1 ; CC(C)(C)OC(=O)Nc1ccc(N2CCC[C@@H](NC(=O)OC(C)(C)C)C2)c([N+](=O)[O-])c1]

A mixture of tert-butyl (4-fluoro-3-nitrophenyl)carbamate Int-2 (1.28 g, 4.99 mmol), (R)-tert-butyl piperidin-3-ylcarbamate (1.00 g, 4.99 mmol) and N,N-diisopropylethylamine (1.74 ml, 9.98 mmol) were heated in acetonitrile (20 ml) at 80° C. overnight. Solvents were removed in vacuum and chromatographic purification (ethyl acetate-hexane) gave Int-3 as orange solid.

### Preparation of Int-5

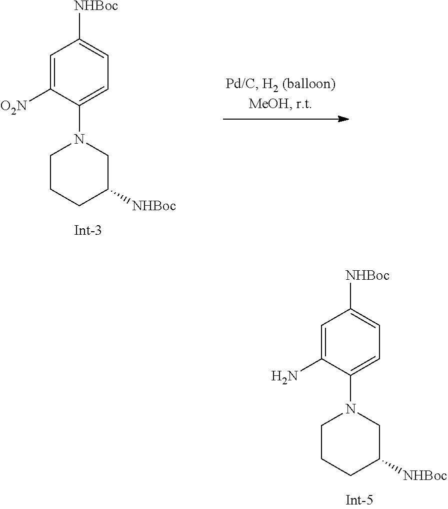 [SMILES: CC(C)(C)OC(=O)Nc1ccc(N2CCC[C@@H](NC(=O)OC(C)(C)C)C2)c([N+](=O)[O-])c1 ; CC(C)(C)OC(=O)Nc1ccc(N2CCC[C@@H](NC(=O)OC(C)(C)C)C2)c(N)c1]

A mixture of Int-3 (1.46 g, 3.34 mmol) and 10% Pd/C (0.71 g, 0.33 mmol, 50% wet) were stirred in methanol (20 ml) under hydrogen (balloon) at r.t for 2 hr. Catalyst was filtered through Celite and solvents were removed in vacuum to give Int-5 as off-white solid. The solid was used in the next step without further purification.

### Preparation of Int-6

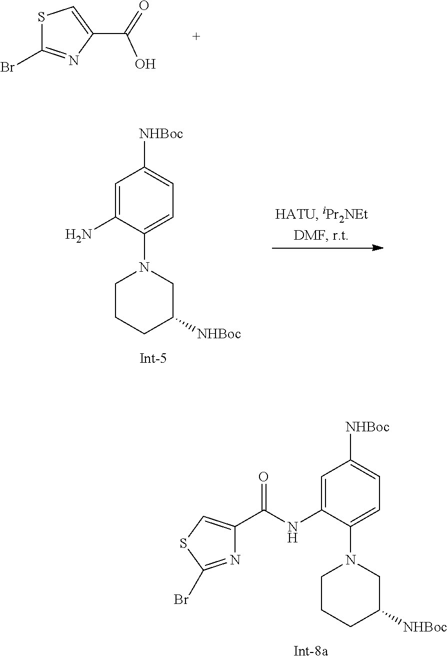 [SMILES: O=C(O)c1csc(Br)n1 ; "+" ; CC(C)(C)OC(=O)Nc1ccc(N2CCC[C@@H](NC(=O)OC(C)(C)C)C2)c(N)c1 ; CC(C)(C)OC(=O)Nc1ccc(N2CCC[C@@H](NC(=O)OC(C)(C)C)C2)c(NC(=O)c2csc(Br)n2)c1]

A mixture of 2-bromothiazole-4-carboxylic acid (180 mg, 0.87 mmol), Int-5 (352 mg, 0.87 mmol), 2-(7-aza-1H-benzotriazole-1-yl)-1,1,3,3-tetramethyluronium hexafluorophosphate (HATU) (354 mg, 0.96 mmol) and N,N-diisopropylethylamine (0.31 ml, 1.74 mmol) were stirred in N,N-dimethylformamide (5 ml) at r.t. overnight. Water and ethyl acetate were added and layers were separated. The separated organic layer was washed with water, dried (MgSO₄) and filtered. Solvents were removed in vacuum and chromatographic purification (ethyl acetate hexane) gave Int-8a as white solid.

Intermediates Bromothiazoles 8b to 8j in Table 1 were Also Prepared Using Similar Procedures for the Preparation of Int-8a.

TABLE 1
(Preparation of Intermediate Bromothiazoles 8a to 8j)

| Intermediates | | |
|---|---|---|
| (#) | Starting Amines | Intermediate Bromothiazoles (structure) |
| 8a | 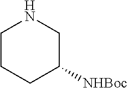 [SMILES: CC(C)(C)OC(=O)N[C@@H]1CCCNC1] | 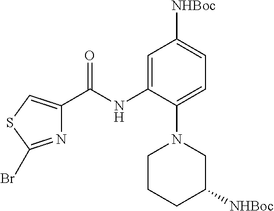 [SMILES: CC(C)(C)OC(=O)Nc1ccc(N2CCC[C@@H](NC(=O)OC(C)(C)C)C2)c(NC(=O)c2csc(Br)n2)c1] |
| 8b | 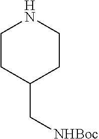 [SMILES: CC(C)(C)OC(=O)NCC1CCNCC1] | 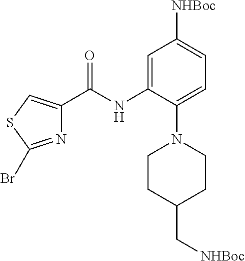 [SMILES: CC(C)(C)OC(=O)NCC1CCN(c2ccc(NC(=O)OC(C)(C)C)cc2NC(=O)c2csc(Br)n2)CC1] |
| 8c | 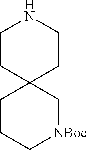 [SMILES: CC(C)(C)OC(=O)N1CCCC2(CCNCC2)C1] | 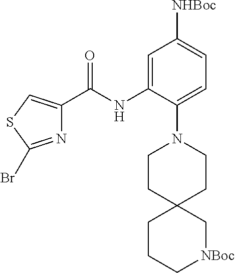 [SMILES: CC(C)(C)OC(=O)Nc1ccc(N2CCC3(CCCN(C(=O)OC(C)(C)C)C3)CC2)c(NC(=O)c2csc(Br)n2)c1] |
| 8d | 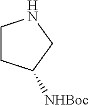 [SMILES: CC(C)(C)OC(=O)N[C@@H]1CCNC1] | 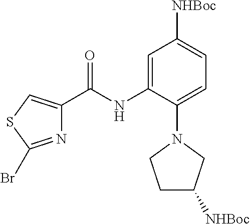 [SMILES: CC(C)(C)OC(=O)Nc1ccc(N2CC[C@@H](NC(=O)OC(C)(C)C)C2)c(NC(=O)c2csc(Br)n2)c1] |
| 8e | 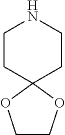 | 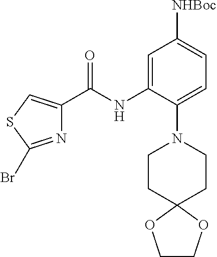 [SMILES: CC(C)(C)OC(=O)Nc1ccc(N2CCC3(CC2)OCCO3)c(NC(=O)c2csc(Br)n2)c1] |
| 8f | 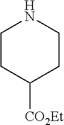 [SMILES: CCOC(=O)C1CCNCC1] | 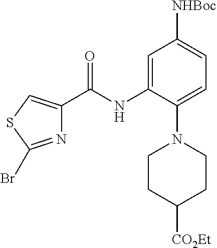 [SMILES: CCOC(=O)C1CCN(c2ccc(NC(=O)OC(C)(C)C)cc2NC(=O)c2csc(Br)n2)CC1] |
| 8g | 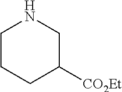 [SMILES: CCOC(=O)C1CCCNC1] | 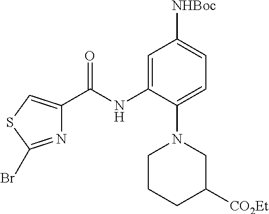 [SMILES: CCOC(=O)C1CCCN(c2ccc(NC(=O)OC(C)(C)C)cc2NC(=O)c2csc(Br)n2)C1] |
| 8h | 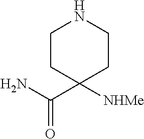 [SMILES: CNC1(C(N)=O)CCNCC1] | 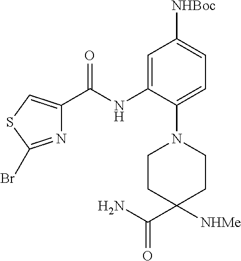 [SMILES: CNC1(C(N)=O)CCN(c2ccc(NC(=O)OC(C)(C)C)cc2NC(=O)c2csc(Br)n2)CC1] |
| 8i | 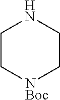 [SMILES: CC(C)(C)OC(=O)N1CCNCC1] | 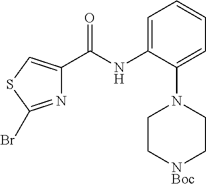 [SMILES: CC(C)(C)OC(=O)N1CCN(c2ccccc2NC(=O)c2csc(Br)n2)CC1] |
| 8j | 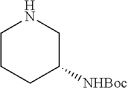 [SMILES: CC(C)(C)OC(=O)N[C@@H]1CCCNC1] | 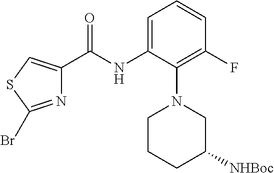 [SMILES: CC(C)(C)OC(=O)N[C@@H]1CCCN(c2c(F)cccc2NC(=O)c2csc(Br)n2)C1] |

### Example 2

### Representative Procedures for the Preparation of Compounds 1-52

### Method A

### Step 1

### Preparation of Int-9

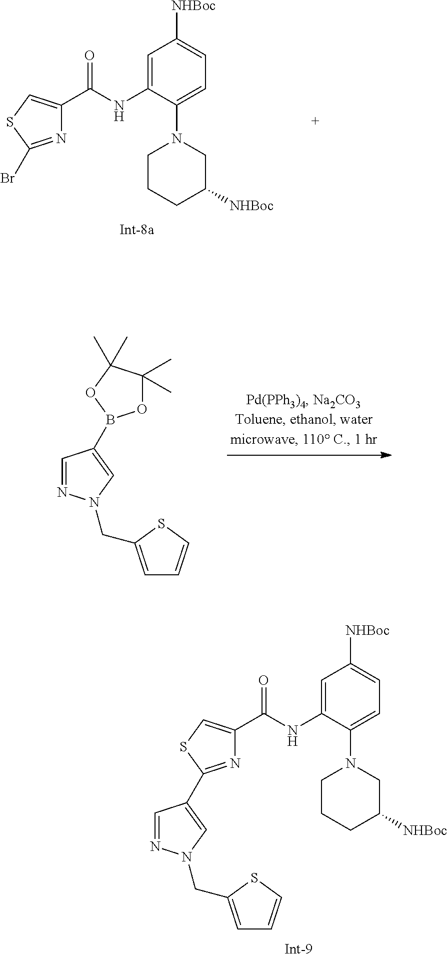 [SMILES: CC(C)(C)OC(=O)Nc1ccc(N2CCC[C@@H](NC(=O)OC(C)(C)C)C2)c(NC(=O)c2csc(Br)n2)c1 ; "+" ; CC1(C)OB(c2cnn(Cc3cccs3)c2)OC1(C)C ; CC(C)(C)OC(=O)Nc1ccc(N2CCC[C@@H](NC(=O)OC(C)(C)C)C2)c(NC(=O)c2csc(-c3cnn(Cc4cccs4)c3)n2)c1]

A mixture of Int-8a (56 mg, 0.094 mmol), 4-(4,4,5,5-tetramethyl-1,3,2-dioxaborolan-2-yl)-1-(thiophen-2-ylmethyl)-1H-pyrazole (33 mg, 0.11 mmol) and sodium carbonate (420 mg, 3.96 mmol) in toluene (1 ml), ethanol (1 ml) and water (0.5 ml) at r.t. were purged with nitrogen gas for 5 min. in a microwave vial. Tetralcistriphenylphosphorous palladium (11 mg, 0.0094 mmol) was added. The mixture was heated in a microwave machine at 110° C. for 1 hr. Water and ethyl acetate were added and layers were separated. The separated aqueous layer was extracted with ethyl acetate. The combined organic layers were dried (MgSO₄) and filtered. Solvents were removed in vacuum and chromatographic purification (ethyl acetate-hexane) gave Int-9 as colorless oil.

### Step 2

### Preparation of Compound 1

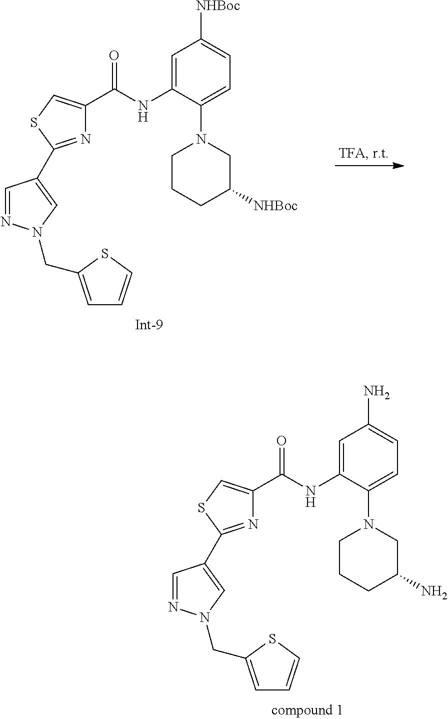 [SMILES: CC(C)(C)OC(=O)Nc1ccc(N2CCC[C@@H](NC(=O)OC(C)(C)C)C2)c(NC(=O)c2csc(-c3cnn(Cc4cccs4)c3)n2)c1 ; Nc1ccc(N2CCC[C@@H](N)C2)c(NC(=O)c2csc(-c3cnn(Cc4cccs4)c3)n2)c1]

Int-9 (54 mg, mmol) was stirred in trifluoroacetic acid (3 ml) at r.t. for 1 hr. Solvents were removed in vacuum. Chromatographic purification [dichloromethane methanol (7N ammonia)] gave compound 1 as colorless oil. LCMS m/e (M+H⁺)=480.3.

### Method B

### Step 1

### Preparation of Int-10

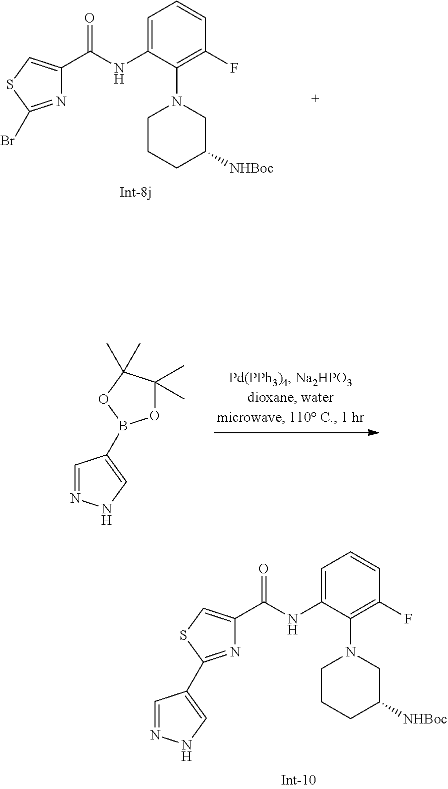 [SMILES: CC(C)(C)OC(=O)N[C@@H]1CCCN(c2c(F)cccc2NC(=O)c2csc(Br)n2)C1 ; "+" ; CC1(C)OB(c2cn[nH]c2)OC1(C)C ; CC(C)(C)OC(=O)N[C@@H]1CCCN(c2c(F)cccc2NC(=O)c2csc(-c3cn[nH]c3)n2)C1]

A mixture of Int-8j (512 mg, 1.03 mmol), 4-(4,4,5,5-tetramethyl-1,3,2-dioxaborolan-2-yl)-1H-pyrazole (239 mg, 1.23 mmol) and disodium hydrogenphosphate (437 mg, 3.08 mmol) in dioxane (6 ml) and water (3 ml) at r.t. were purged with nitrogen gas for 5 min in a microwave vial. Tetrakistriphenylphosphorous palladium (119 mg, 0.103 mmol) was added. The mixture was heated in a microwave machine at 110° C. for 1 hr. Water and ethyl acetate were added and layers were separated. The separated aqueous layer was extracted with ethyl acetate. The combined organic layers were dried (MgSO₄) and filtered. Solvents were removed in vacuum and chromatographic purification (ethyl acetate-hexane) gave Int-10 as yellow foam.

### Step 2

### Preparation of Int-11

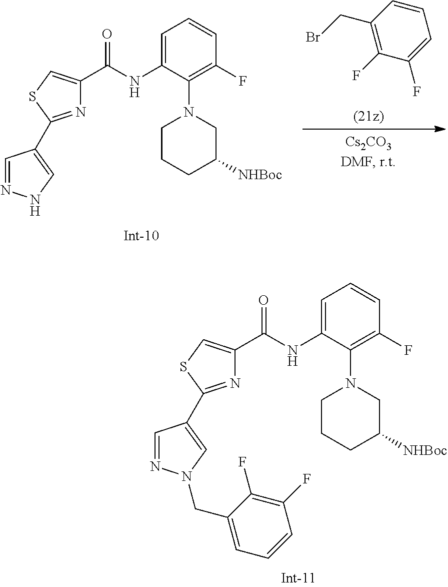 [SMILES: CC(C)(C)OC(=O)N[C@@H]1CCCN(c2c(F)cccc2NC(=O)c2csc(-c3cn[nH]c3)n2)C1 ; Fc1cccc(CBr)c1F ; CC(C)(C)OC(=O)N[C@@H]1CCCN(c2c(F)cccc2NC(=O)c2csc(-c3cnn(Cc4cccc(F)c4F)c3)n2)C1]

A mixture of 1-(bromomethyl)-2,3-difluorobenzene 21y (20 mg, 0.095 mmol), Int-10 (42 mg, 0.086 mmol) and cesium carbonate (84 mg, 0.26 mmol) were stirred in N,N-dimethylformamide (2 ml) at r.t. overnight. Ethyl acetate and water were added and layers were separated. The separated organic layer was dried (MgSO₄) and filtered. Solvents were removed in vacuum and chromatographic purification (ethyl acetate-hexane) gave Int-11 as colorless oil.

### Step 3

### Preparation of Compound 52

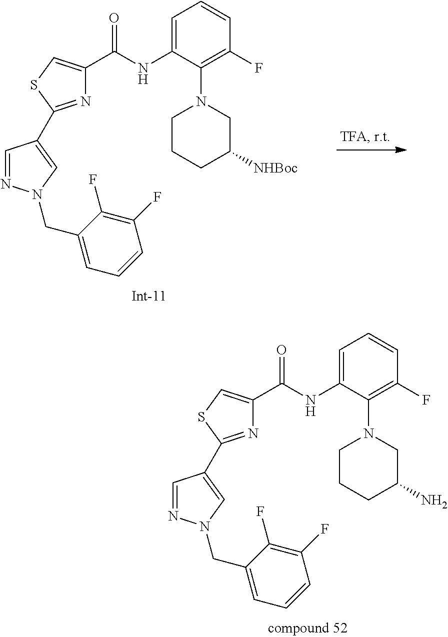 [SMILES: CC(C)(C)OC(=O)N[C@@H]1CCCN(c2c(F)cccc2NC(=O)c2csc(-c3cnn(Cc4cccc(F)c4F)c3)n2)C1 ; N[C@@H]1CCCN(c2c(F)cccc2NC(=O)c2csc(-c3cnn(Cc4cccc(F)c4F)c3)n2)C1]

Int-11 (41 mg, 0.067 mmol) was stirred in trifluoroacetic acid (3 ml) at r.t. for 1 hr. Solvents were removed in vacuum. Chromatographic purification [dichloromethane methanol (7N ammonia)] gave compound 52 as colorless oil. LCMS m/e (M+H⁺)=513.3.

### Preparation of Compound 44

### Step 1

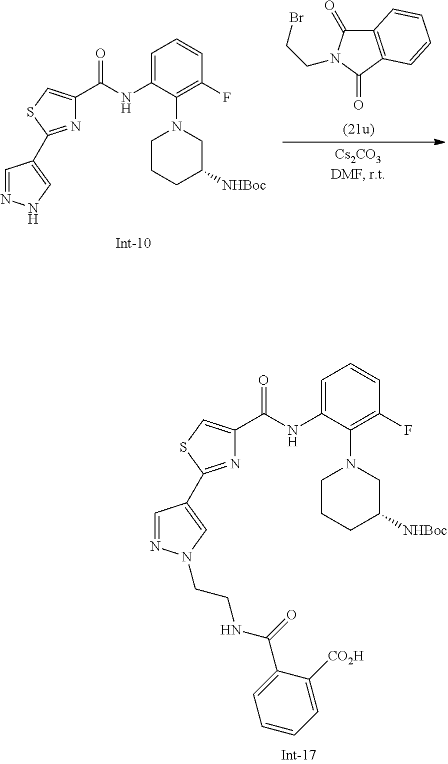 [SMILES: CC(C)(C)OC(=O)N[C@@H]1CCCN(c2c(F)cccc2NC(=O)c2csc(-c3cn[nH]c3)n2)C1 ; O=C1c2ccccc2C(=O)N1CCBr ; CC(C)(C)OC(=O)N[C@@H]1CCCN(c2c(F)cccc2NC(=O)c2csc(-c3cnn(CCNC(=O)c4ccccc4C(=O)O)c3)n2)C1]

A mixture of 2-(2-bromoethyl)isoindoline-1,3-dione 21u (30 mg, 0.12 mmol), Int-10 (58 mg, 0.099 mmol) and cesium carbonate (97 mg, 0.30 mmol) were stirred in N,N-dimethylformamide (2 ml) at r.t. overnight. Ethyl acetate and water were added and layers were separated. The separated organic layer was dried (MgSO₄) and filtered. Solvents were removed in vacuum and chromatographic purification (ethyl acetate-hexane) gave Int-17 as colorless oil. The phthalimide group was hydrolyzed during work-up.

### Step 2

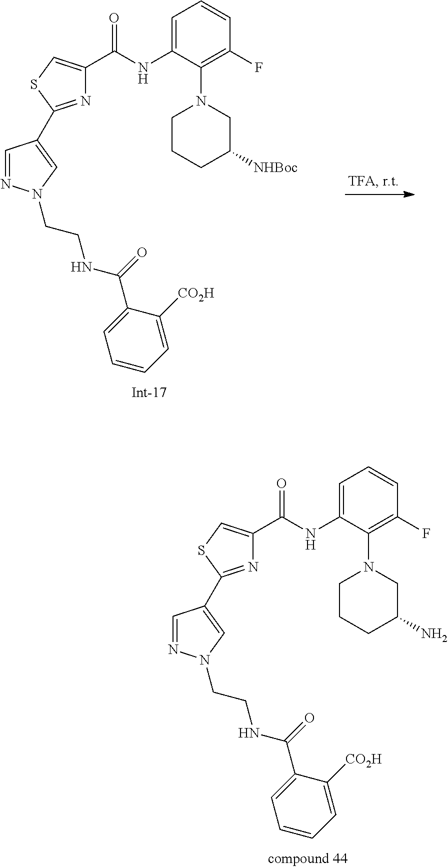 [SMILES: CC(C)(C)OC(=O)N[C@@H]1CCCN(c2c(F)cccc2NC(=O)c2csc(-c3cnn(CCNC(=O)c4ccccc4C(=O)O)c3)n2)C1 ; N[C@@H]1CCCN(c2c(F)cccc2NC(=O)c2csc(-c3cnn(CCNC(=O)c4ccccc4C(=O)O)c3)n2)C1]

A solution of Int-17 (51 mg, 0.076 innaol) in trifluoroacetic acid (3 ml) was stirred at r.t. for 3 hr. Solvents were removed in vacuum. The residue was dissolved in minimum methanol and hydrochloric acid (1M in diethyl ether) was added. The solid was filtered to give compound 44 as white solid. LCMS (M+575.3.

### Preparation of Compound 46 from Compound 45

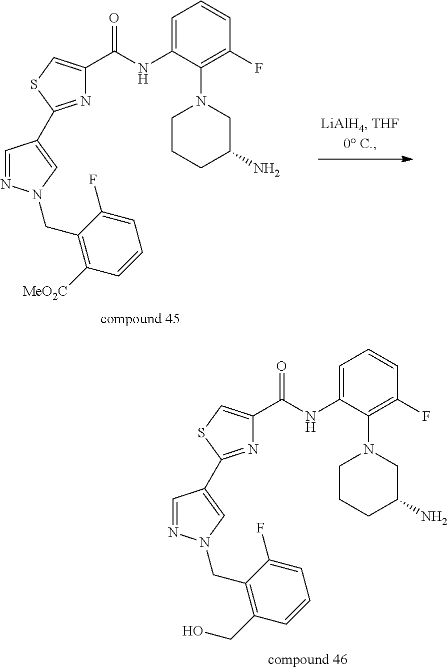 [SMILES: COC(=O)c1cccc(F)c1Cn1cc(-c2nc(C(=O)Nc3cccc(F)c3N3CCC[C@@H](N)C3)cs2)cn1 ; N[C@@H]1CCCN(c2c(F)cccc2NC(=O)c2csc(-c3cnn(Cc4c(F)cccc4CO)c3)n2)C1]

To a solution of compound 45 (32 mg, 0.058 mmol) in tetrahydrofuran (3 ml) at 0° C. under nitrogen, a solution of lithium aluminum hydride (58 μl, 0.058 mmol, 1.0 M in THF) was added. The mixture was stirred at 0° C. for 2 hr before it was quenched with saturated sodium potassium tartrate solution. Ethyl acetate was added and layers were separated. The separated aqueous layer was extracted with ethyl acetate. The combined organic layers were dried (MgSO₄) and filtered. Solvents were removed in vacuum and chromatographic purification [ethyl acetate-methanol (7N, ammonia)] gave compound 46 as colorless oil. LCMS m/e (M+H⁺)=5253.

### Preparation of Compound 50

### Step 1

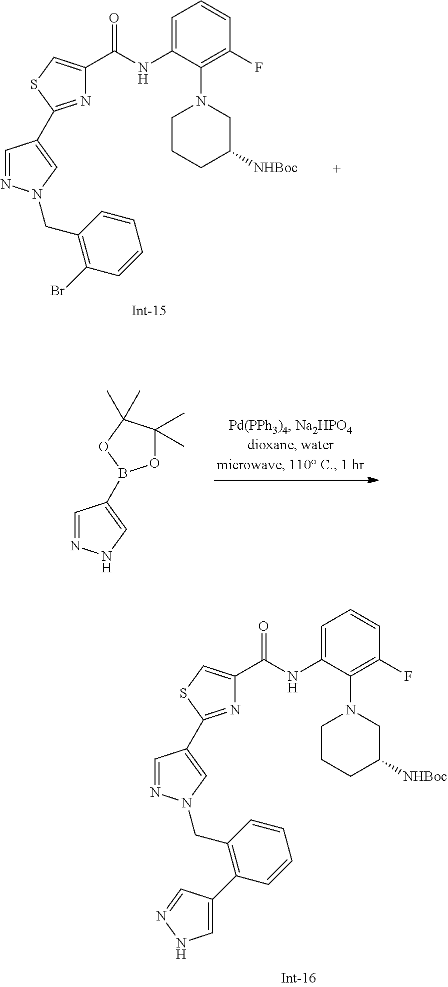 [SMILES: CC(C)(C)OC(=O)N[C@@H]1CCCN(c2c(F)cccc2NC(=O)c2csc(-c3cnn(Cc4ccccc4Br)c3)n2)C1 ; "+" ; CC1(C)OB(c2cn[nH]c2)OC1(C)C ; CC(C)(C)OC(=O)N[C@@H]1CCCN(c2c(F)cccc2NC(=O)c2csc(-c3cnn(Cc4ccccc4-c4cn[nH]c4)c3)n2)C1]

A mixture of Int-15 (50 mg, 0.076 mmol), 4-(4,4,5,5-tetramethyl-1,3,2-dioxaborolan-2-yl)-1H-pyrazole (22 mg, 0.114 mmol) and disodium hydrogenphosphate (32 mg, 0.23 mmol) in dioxane (2 ml) and water (1 ml) at r.t. were purged with nitrogen gas for 5 min in a microwave vial. Tetrakistriphenylphosphorous palladium (9 mg, 0.0076 mmol) was added. The mixture was heated in a microwave machine at 110° C. for 1 hr. Water and ethyl acetate were added and layers were separated. The separated aqueous layer was extracted with ethyl acetate. The combined organic layers were dried (MgSO₄) and filtered. Solvents were removed in vacuum and chromatographic purification (ethyl acetate-hexane) gave Int-16 as colorless oil.

### Step 2

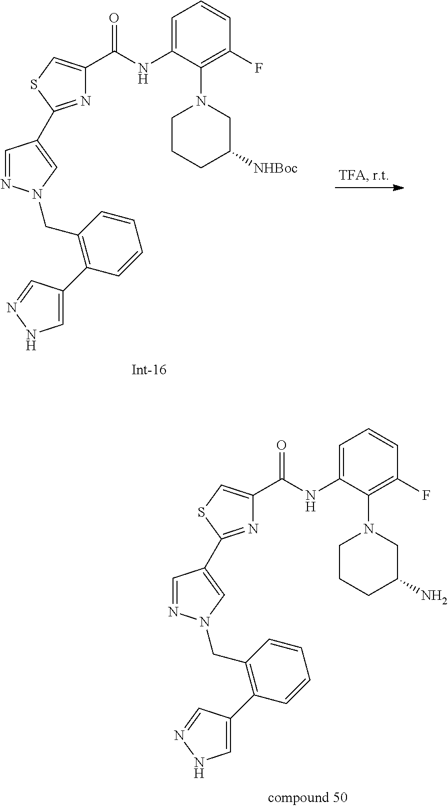 [SMILES: CC(C)(C)OC(=O)N[C@@H]1CCCN(c2c(F)cccc2NC(=O)c2csc(-c3cnn(Cc4ccccc4-c4cn[nH]c4)c3)n2)C1 ; N[C@@H]1CCCN(c2c(F)cccc2NC(=O)c2csc(-c3cnn(Cc4ccccc4-c4cn[nH]c4)c3)n2)C1]

Int-16 (34 mg, 0.053 mmol) was stirred in trifluoroacetic acid (3 ml) at r.t. for 1 hr. Solvents were removed in vacuum. Chromatographic purification [dichloromethane methanol (7N ammonia)] gave compound 50 as colorless oil. LCMS role (M+H⁺)=543.1.

### Preparation of Compound 51

### Step 1

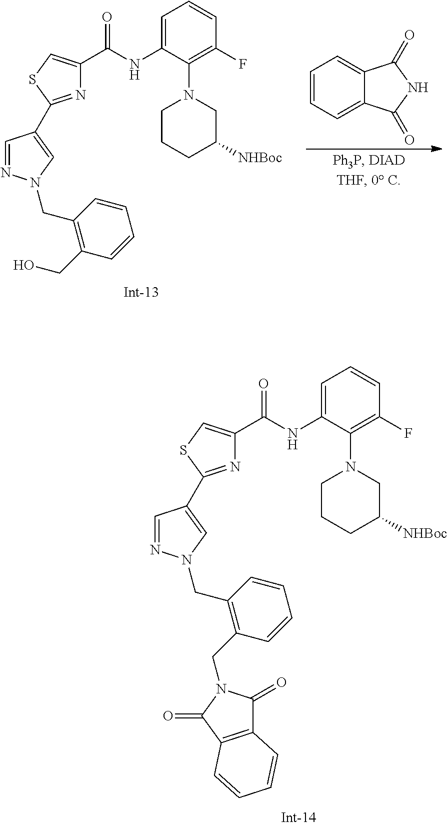 [SMILES: CC(C)(C)OC(=O)N[C@@H]1CCCN(c2c(F)cccc2NC(=O)c2csc(-c3cnn(Cc4ccccc4CO)c3)n2)C1 ; O=C1NC(=O)c2ccccc21 ; CC(C)(C)OC(=O)N[C@@H]1CCCN(c2c(F)cccc2NC(=O)c2csc(-c3cnn(Cc4ccccc4CN4C(=O)c5ccccc5C4=O)c3)n2)C1]

To a solution of triphenylphosphine (86 mg, 0.14 mmol) in tetrahydrofuran (1 ml) at 0° C. under nitrogen, a solution of diisopropyl azodicarboxylate (DIAD) (57 mg, 0.28 mmol) in tetrahydrofuran (1 ml) was added. The mixture was stirred at 0° C. for 5 min. before a solution of Int-13 (86 mg, 0.14 mmol) in tetrahydrofuran (1 ml) was added. The mixture was stirred at 0° C. for another 5 min. and a solution of phthalimide (42 mg, 0.28 mmol) in tetrahydrofuran (1 ml) was added. The mixture was stirred at 0° C. for a further 2 hr. Solvents were removed in vacuum and chromatographic purification [ethyl acetate-methanol (7N, ammonia)] gave Int-14 as colorless oil.

### Step 2

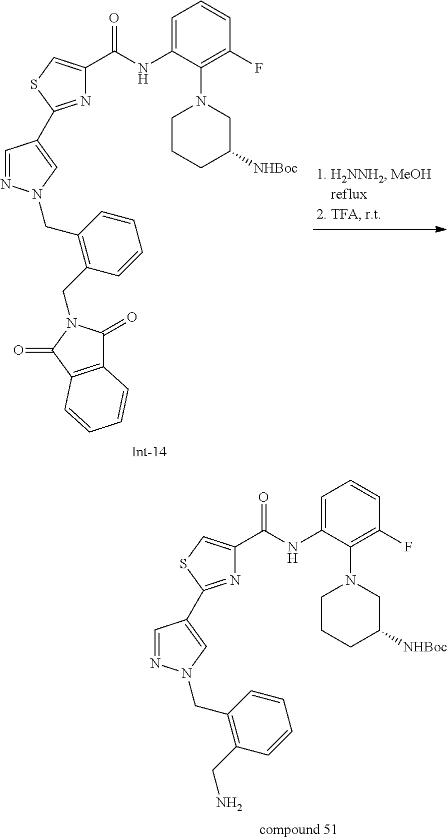 [SMILES: CC(C)(C)OC(=O)N[C@@H]1CCCN(c2c(F)cccc2NC(=O)c2csc(-c3cnn(Cc4ccccc4CN4C(=O)c5ccccc5C4=O)c3)n2)C1 ; CC(C)(C)OC(=O)N[C@@H]1CCCN(c2c(F)cccc2NC(=O)c2csc(-c3cnn(Cc4ccccc4CN)c3)n2)C1]

To a solution of Int-14 (75 mg, 0.102 mmol) and hydrazine (33 mg, 1.02 mmol) in methanol (2 ml) was heated at reflux for 2 hr. Solvents were removed in vacuum. Trifluoroacetic acid (5 ml) was added and the mixture was stirred at r.t. for 2 hr. Solvents were removed in vacuum. Chromatographic purification [dichloromethane-methanol (7N ammonia)] gave compound 51 as colorless oil. LCMS node (M+H⁺)=506.1.

TABLE 2
(Boronate Esters 20a to 20g)

| | |
|---|---|
| 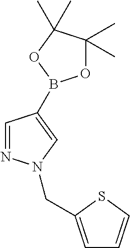 [SMILES: CC1(C)OB(c2cnn(Cc3cccs3)c2)OC1(C)C] | 20a |
| 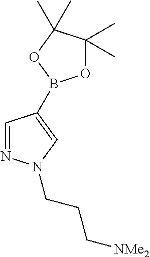 [SMILES: CN(C)CCCn1cc(B2OC(C)(C)C(C)(C)O2)cn1] | 20b |
| 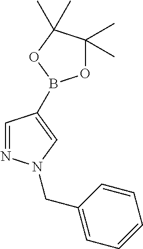 [SMILES: CC1(C)OB(c2cnn(Cc3ccccc3)c2)OC1(C)C] | 20c |
| 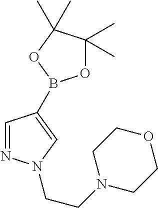 [SMILES: CC1(C)OB(c2cnn(CCN3CCOCC3)c2)OC1(C)C] | 20d |
| 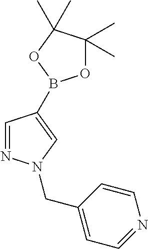 [SMILES: CC1(C)OB(c2cnn(Cc3ccncc3)c2)OC1(C)C] | 20e |
| 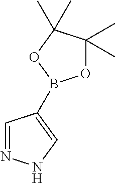 | 20f |
| 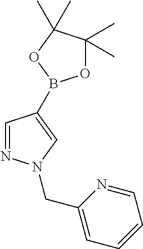 [SMILES: CC1(C)OB(c2cnn(Cc3ccccn3)c2)OC1(C)C] | 20g |

TABLE 3
(Alkylbromides 21a to 21z)

| | |
|---|---|
| 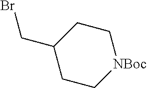 [SMILES: CC(C)(C)OC(=O)N1CCC(CBr)CC1] | 21a |
| 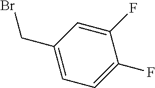 | 21b |
| 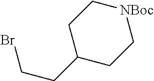 [SMILES: CC(C)(C)OC(=O)N1CCC(CCBr)CC1] | 21c |
| 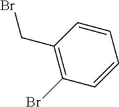 | 21d |
| 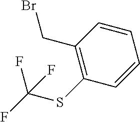 [SMILES: FC(F)(F)Sc1ccccc1CBr] | 21e |
| 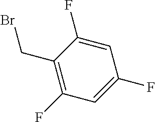 [SMILES: Fc1cc(F)c(CBr)c(F)c1] | 21f |
| 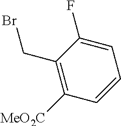 [SMILES: COC(=O)c1cccc(F)c1CBr] | 21g |
| 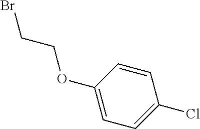 [SMILES: Clc1ccc(OCCBr)cc1] | 21h |
| 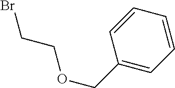 [SMILES: BrCCOCc1ccccc1] | 21i |
| 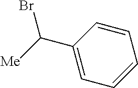 | 21j |
| 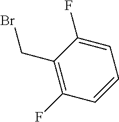 | 21k |
| 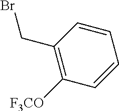 [SMILES: FC(F)(F)Oc1ccccc1CBr] | 21l |
| 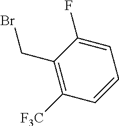 [SMILES: Fc1cccc(C(F)(F)F)c1CBr] | 21m |
| 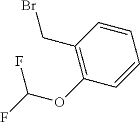 [SMILES: FC(F)Oc1ccccc1CBr] | 21n |
| 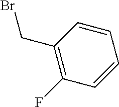 | 21o |
| 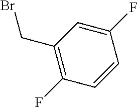 | 21p |
| 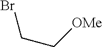 | 21q |
| 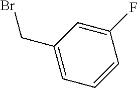 | 21r |
| 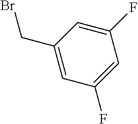 | 21s |
| 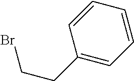 | 21t |
| 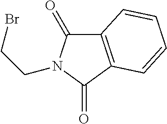 [SMILES: O=C1c2ccccc2C(=O)N1CCBr] | 21u |
| 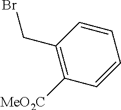 [SMILES: COC(=O)c1ccccc1CBr] | 21v |
| 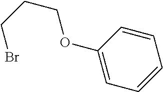 [SMILES: BrCCCOc1ccccc1] | 21w |
| 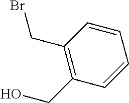 | 21x |
| 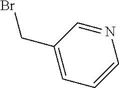 | 21y |
| 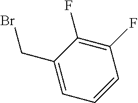 | 21z |

Table 4 below lists representative compounds of the invention with activity data whereby the IC₅₀ values are rated “A”, “B,” “C,” or “D.” The IC₅₀ values are rated “A” for IC₅₀ values in the range of 1 nM to 50 nM, “B” for IC₅₀ values in the range from 51 nM to 250 nM, “C” for IC₅₀ values in the range from 251 nM to 1 μM, and “D” for IC₅₀ values greater than 1 μM.

Compounds 1 to 52 were prepared by either method A or B as described above.

TABLE 4
(Summary of Compounds 1 to 52)

| | | Alkylbromides | | | | |
|---|---|---|---|---|---|---|
| | | (Table 3) or | | | | |
| | Intermediate | Boronate | | | PDK1 | LCMS |
| Cpd | Bromothiazoles | Esters | | Compound | IC₅₀ | M + H⁺ |
| (#) | (Table 1) | (Table 2) | Method | (Structure) | (nM) | (observed) |
| 1 | 8a | 20a | A | 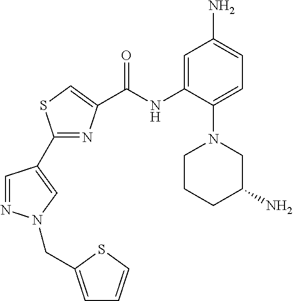 [SMILES: Nc1ccc(N2CCC[C@@H](N)C2)c(NC(=O)c2csc(-c3cnn(Cc4cccs4)c3)n2)c1] | 7 | 480.3 |
| 2 | 8a | 20b | A | 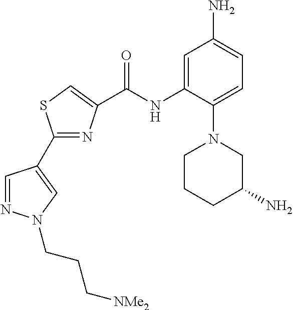 [SMILES: CN(C)CCCn1cc(-c2nc(C(=O)Nc3cc(N)ccc3N3CCC[C@@H](N)C3)cs2)cn1] | C | 469.3 |
| 3 | 8a | 20c | A | 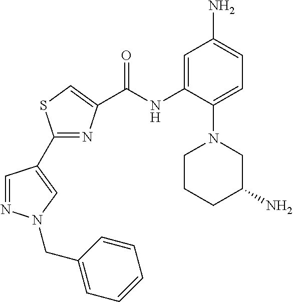 [SMILES: Nc1ccc(N2CCC[C@@H](N)C2)c(NC(=O)c2csc(-c3cnn(Cc4ccccc4)c3)n2)c1] | A | 474.3 |
| 4 | 8a | 20d | A | 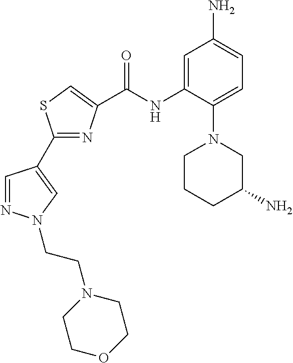 [SMILES: Nc1ccc(N2CCC[C@@H](N)C2)c(NC(=O)c2csc(-c3cnn(CCN4CCOCC4)c3)n2)c1] | C | 497.3 |
| 5 | 8h | 20a | A | 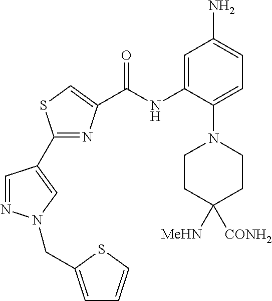 [SMILES: CNC1(C(N)=O)CCN(c2ccc(N)cc2NC(=O)c2csc(-c3cnn(Cc4cccs4)c3)n2)CC1] | 81 | 537.3 |
| 6 | 8b | 20a | A | 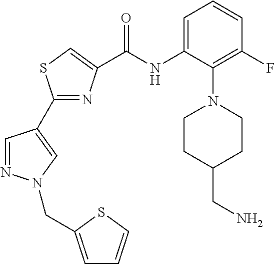 [SMILES: NCC1CCN(c2c(F)cccc2NC(=O)c2csc(-c3cnn(Cc4cccs4)c3)n2)CC1] | B | 497.3 |
| 7 | 8c | 20a | A | 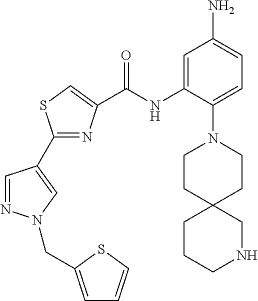 [SMILES: Nc1ccc(N2CCC3(CCCNC3)CC2)c(NC(=O)c2csc(-c3cnn(Cc4cccs4)c3)n2)c1] | 60 | 534.3 |
| 8 | 8b | 20a | A | 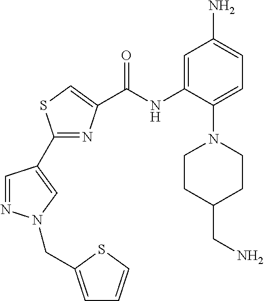 [SMILES: NCC1CCN(c2ccc(N)cc2NC(=O)c2csc(-c3cnn(Cc4cccs4)c3)n2)CC1] | A | 494.3 |
| 9 | 8a | 20e | A | 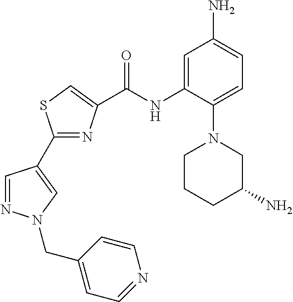 [SMILES: Nc1ccc(N2CCC[C@@H](N)C2)c(NC(=O)c2csc(-c3cnn(Cc4ccncc4)c3)n2)c1] | B | 475.3 |
| 10 | 8d | 20a | A | 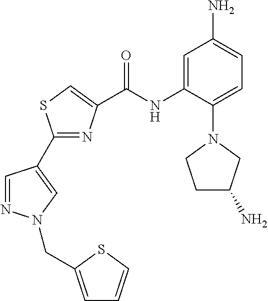 [SMILES: Nc1ccc(N2CC[C@@H](N)C2)c(NC(=O)c2csc(-c3cnn(Cc4cccs4)c3)n2)c1] | A | 466.3 |
| 11 | 8g | 20a | A | 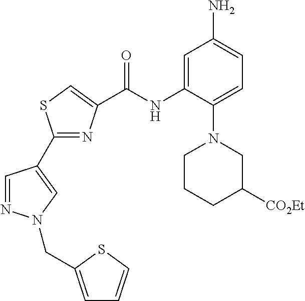 [SMILES: CCOC(=O)C1CCCN(c2ccc(N)cc2NC(=O)c2csc(-c3cnn(Cc4cccs4)c3)n2)C1] | D | 537.3 |
| 12 | 8f | 20a | A | 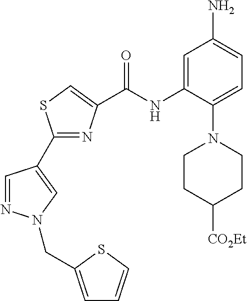 [SMILES: CCOC(=O)C1CCN(c2ccc(N)cc2NC(=O)c2csc(-c3cnn(Cc4cccs4)c3)n2)CC1] | C | 537.3 |
| 13 | 8a | 20g | B | 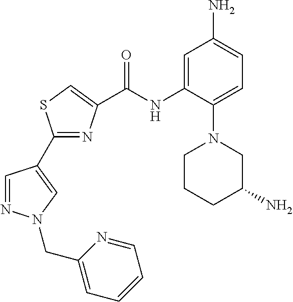 [SMILES: Nc1ccc(N2CCC[C@@H](N)C2)c(NC(=O)c2csc(-c3cnn(Cc4ccccn4)c3)n2)c1] | A | 475.3 |
| 14 | 8a | 21b | B | 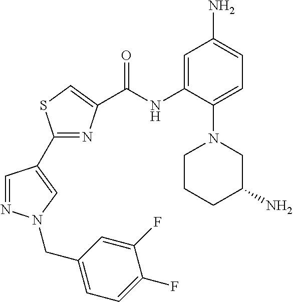 [SMILES: Nc1ccc(N2CCC[C@@H](N)C2)c(NC(=O)c2csc(-c3cnn(Cc4ccc(F)c(F)c4)c3)n2)c1] | A | 510.3 |
| 15 | 8e | 21r | B | 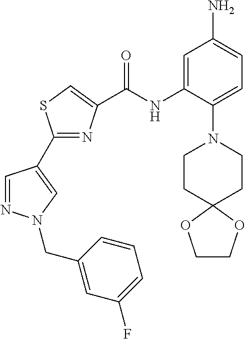 [SMILES: Nc1ccc(N2CCC3(CC2)OCCO3)c(NC(=O)c2csc(-c3cnn(Cc4cccc(F)c4)c3)n2)c1] | A | 535.3 |
| 16 | 8a | 21s | B | 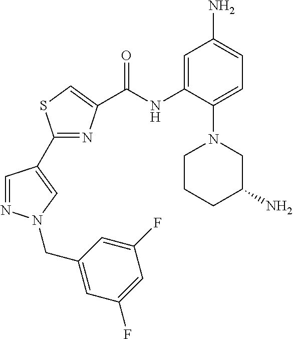 [SMILES: Nc1ccc(N2CCC[C@@H](N)C2)c(NC(=O)c2csc(-c3cnn(Cc4cc(F)cc(F)c4)c3)n2)c1] | A | 510.3 |
| 17 | 8a | 21t | B | 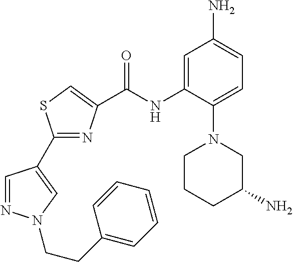 [SMILES: Nc1ccc(N2CCC[C@@H](N)C2)c(NC(=O)c2csc(-c3cnn(CCc4ccccc4)c3)n2)c1] | A | 488.3 |
| 18 | 8a | 21y | B | 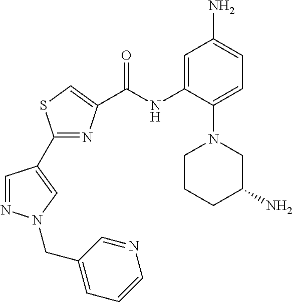 [SMILES: Nc1ccc(N2CCC[C@@H](N)C2)c(NC(=O)c2csc(-c3cnn(Cc4cccnc4)c3)n2)c1] | A | 475.3 |
| 19 | 8a | 21r | B | 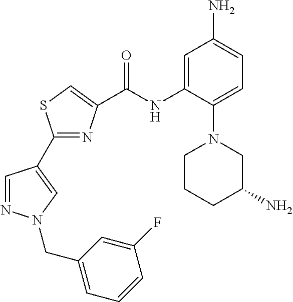 [SMILES: Nc1ccc(N2CCC[C@@H](N)C2)c(NC(=O)c2csc(-c3cnn(Cc4cccc(F)c4)c3)n2)c1] | A | 492.3 |
| 20 | 8c | 20g | A | 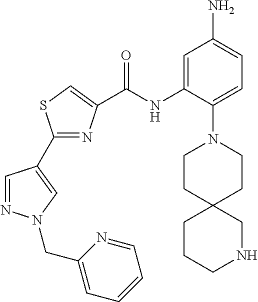 [SMILES: Nc1ccc(N2CCC3(CCCNC3)CC2)c(NC(=O)c2csc(-c3cnn(Cc4ccccn4)c3)n2)c1] | B | 529.3 |
| 21 | 8a | 21o | B | 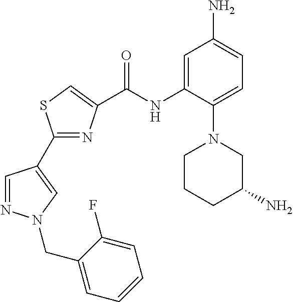 [SMILES: Nc1ccc(N2CCC[C@@H](N)C2)c(NC(=O)c2csc(-c3cnn(Cc4ccccc4F)c3)n2)c1] | A | 492.3 |
| 22 | 8a | 21p | B | 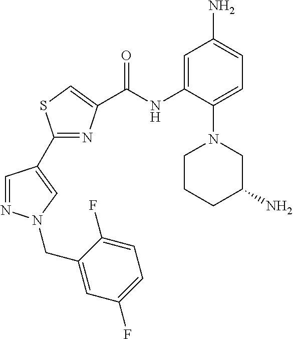 [SMILES: Nc1ccc(N2CCC[C@@H](N)C2)c(NC(=O)c2csc(-c3cnn(Cc4cc(F)ccc4F)c3)n2)c1] | A | 510.3 |
| 23 | 8j | 20a | A | 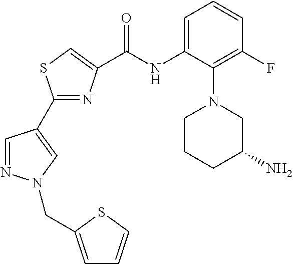 [SMILES: N[C@@H]1CCCN(c2c(F)cccc2NC(=O)c2csc(-c3cnn(Cc4cccs4)c3)n2)C1] | A | 483.3 |
| 24 | 8b | 20g | A | 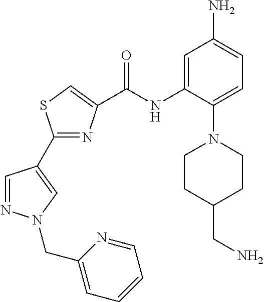 [SMILES: NCC1CCN(c2ccc(N)cc2NC(=O)c2csc(-c3cnn(Cc4ccccn4)c3)n2)CC1] | B | 489.3 |
| 25 | 8j | 20c | A | 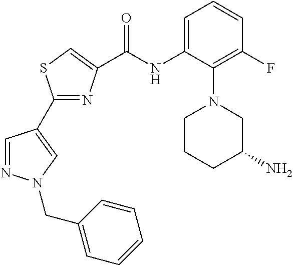 [SMILES: N[C@@H]1CCCN(c2c(F)cccc2NC(=O)c2csc(-c3cnn(Cc4ccccc4)c3)n2)C1] | B | 477.3 |
| 26 | 8a | 21q | B | 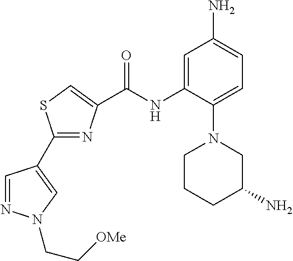 [SMILES: COCCn1cc(-c2nc(C(=O)Nc3cc(N)ccc3N3CCC[C@@H](N)C3)cs2)cn1] | A | 442.2 |
| 27 | 8j | 20g | A | 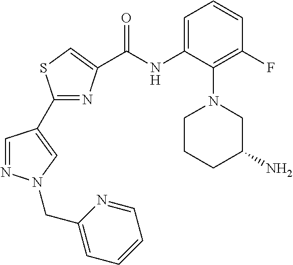 [SMILES: N[C@@H]1CCCN(c2c(F)cccc2NC(=O)c2csc(-c3cnn(Cc4ccccn4)c3)n2)C1] | B | 478.3 |
| 29 | 8d | 20g | A | 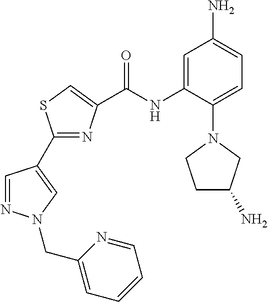 [SMILES: Nc1ccc(N2CC[C@@H](N)C2)c(NC(=O)c2csc(-c3cnn(Cc4ccccn4)c3)n2)c1] | B | 461.3 |
| 30 | 8a | 21j | B | 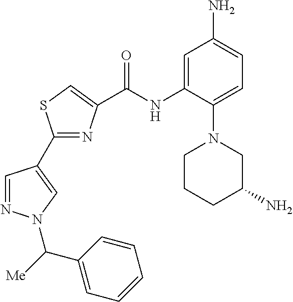 [SMILES: CC(c1ccccc1)n1cc(-c2nc(C(=O)Nc3cc(N)ccc3N3CCC[C@@H](N)C3)cs2)cn1] | B | 488.3 |
| 31 | 8j | 21y | B | 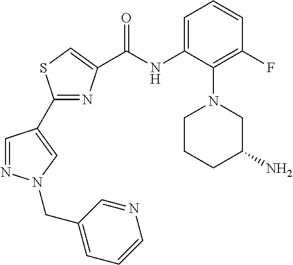 [SMILES: N[C@@H]1CCCN(c2c(F)cccc2NC(=O)c2csc(-c3cnn(Cc4cccnc4)c3)n2)C1] | B | 478.3 |
| 32 | 8j | 21k | B | 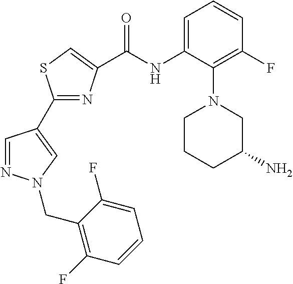 [SMILES: N[C@@H]1CCCN(c2c(F)cccc2NC(=O)c2csc(-c3cnn(Cc4c(F)cccc4F)c3)n2)C1] | B | 513.3 |
| 33 | 8j | 21l | B | 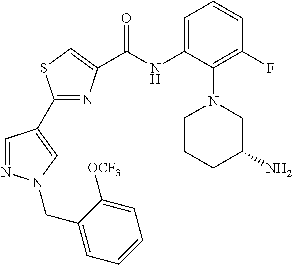 [SMILES: N[C@@H]1CCCN(c2c(F)cccc2NC(=O)c2csc(-c3cnn(Cc4ccccc4OC(F)(F)F)c3)n2)C1] | D | 561.3 |
| 34 | 8e | 20a | A | 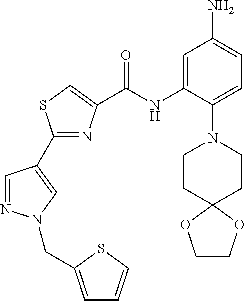 [SMILES: Nc1ccc(N2CCC3(CC2)OCCO3)c(NC(=O)c2csc(-c3cnn(Cc4cccs4)c3)n2)c1] | B | 523.3 |
| 35 | 8j | 21m | B | 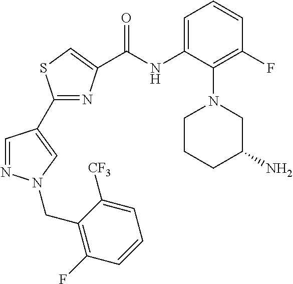 [SMILES: N[C@@H]1CCCN(c2c(F)cccc2NC(=O)c2csc(-c3cnn(Cc4c(F)cccc4C(F)(F)F)c3)n2)C1] | D | 563.3 |
| 36 | 8j | 21n | B | 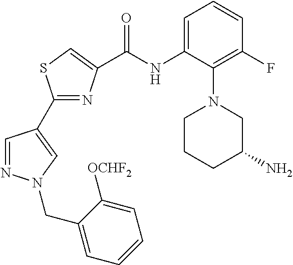 [SMILES: N[C@@H]1CCCN(c2c(F)cccc2NC(=O)c2csc(-c3cnn(Cc4ccccc4OC(F)F)c3)n2)C1] | C | 543.3 |
| 37 | 8j | 21e | B | 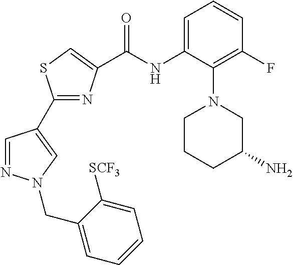 | D | 577.3 |
| 38 | 8j | 21f | B | 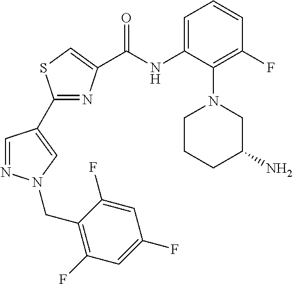 [SMILES: N[C@@H]1CCCN(c2c(F)cccc2NC(=O)c2csc(-c3cnn(Cc4c(F)cc(F)cc4F)c3)n2)C1] | C | 531.3 |
| 39 | 8i | 21p | B | 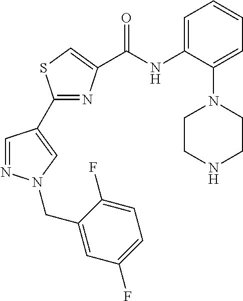 [SMILES: O=C(Nc1ccccc1N1CCNCC1)c1csc(-c2cnn(Cc3cc(F)ccc3F)c2)n1] | C | 481.3 |
| 40 | 8j | 21i | B | 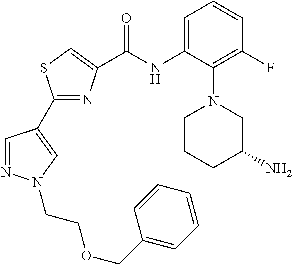 [SMILES: N[C@@H]1CCCN(c2c(F)cccc2NC(=O)c2csc(-c3cnn(CCOCc4ccccc4)c3)n2)C1] | C | 521.3 |
| 41 | 8j | 21h | B | 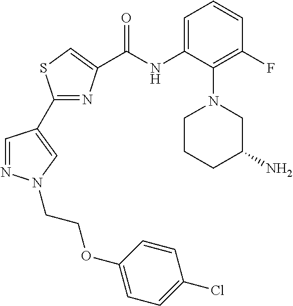 [SMILES: N[C@@H]1CCCN(c2c(F)cccc2NC(=O)c2csc(-c3cnn(CCOc4ccc(Cl)cc4)c3)n2)C1] | D | 509.3 |
| 42 | 8j | 21v | B | 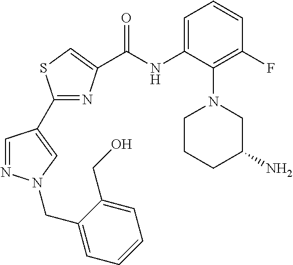 [SMILES: N[C@@H]1CCCN(c2c(F)cccc2NC(=O)c2csc(-c3cnn(Cc4ccccc4CO)c3)n2)C1] | B | 507.3 |
| 43 | 8j | 21w | B | 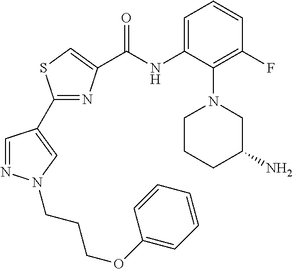 [SMILES: N[C@@H]1CCCN(c2c(F)cccc2NC(=O)c2csc(-c3cnn(CCCOc4ccccc4)c3)n2)C1] | D | 521.3 |
| 44 | 8j | 21u | B | 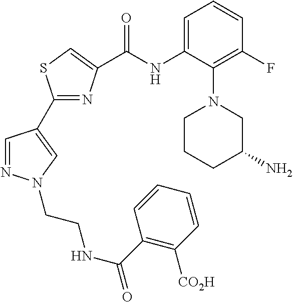 [SMILES: N[C@@H]1CCCN(c2c(F)cccc2NC(=O)c2csc(-c3cnn(CCNC(=O)c4ccccc4C(=O)O)c3)n2)C1] | C | 575.3 |
| 45 | 8j | 21g | B | 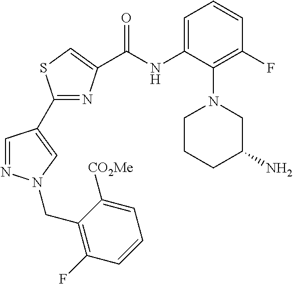 [SMILES: COC(=O)c1cccc(F)c1Cn1cc(-c2nc(C(=O)Nc3cccc(F)c3N3CCC[C@@H](N)C3)cs2)cn1] | C | 553.3 |
| 46 | 8j | 21g | B | 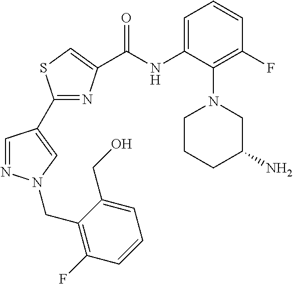 [SMILES: N[C@@H]1CCCN(c2c(F)cccc2NC(=O)c2csc(-c3cnn(Cc4c(F)cccc4CO)c3)n2)C1] | B | 525.3 |
| 47 | 8j | 21a | B | 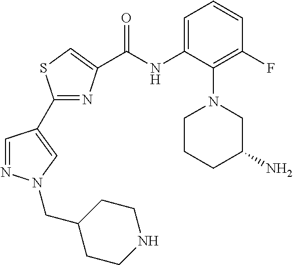 [SMILES: N[C@@H]1CCCN(c2c(F)cccc2NC(=O)c2csc(-c3cnn(CC4CCNCC4)c3)n2)C1] | D | 484.3 |
| 48 | 8j | 21c | B | 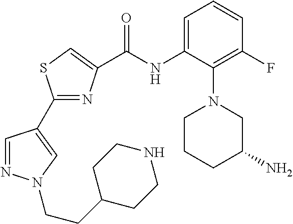 [SMILES: N[C@@H]1CCCN(c2c(F)cccc2NC(=O)c2csc(-c3cnn(CCC4CCNCC4)c3)n2)C1] | D | 466.3 |
| 49 | 8j | 21d | B | 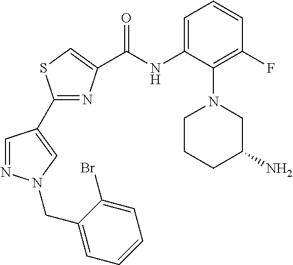 [SMILES: N[C@@H]1CCCN(c2c(F)cccc2NC(=O)c2csc(-c3cnn(Cc4ccccc4Br)c3)n2)C1] | C | 557 |
| 50 | 8j | 21d | B | 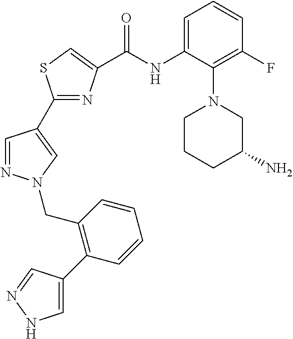 [SMILES: N[C@@H]1CCCN(c2c(F)cccc2NC(=O)c2csc(-c3cnn(Cc4ccccc4-c4cn[nH]c4)c3)n2)C1] | D | 543.1 |
| 51 | 8j | 21x | B | 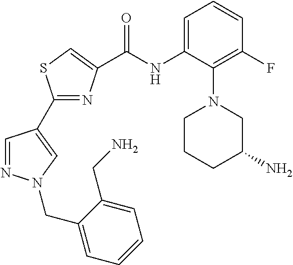 [SMILES: NCc1ccccc1Cn1cc(-c2nc(C(=O)Nc3cccc(F)c3N3CCC[C@@H](N)C3)cs2)cn1] | C | 506.1 |
| 52 | 8j | 21z | B | 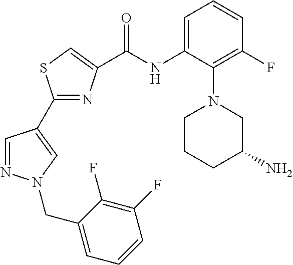 [SMILES: N[C@@H]1CCCN(c2c(F)cccc2NC(=O)c2csc(-c3cnn(Cc4cccc(F)c4F)c3)n2)C1] | B | 513.3 |

TABLE 5
Chemical Names for Compounds 1 to 52

| Compounds | Chemical Names |
|---|---|
| 1 | N-[5-amino-2-(3(R)-amino-1-piperidinyl)phenyl]-2-[1-[3- |
| | (dimethylamino)propyl]-1H-pyrazol-4-yl]-4-thiazolecarboxamide |
| 2 | N-[5-amino-2-(3(R)-amino-1-piperidinyl)phenyl]-2-[1-(2-thienylmethyl)-1H- |
| | pyrazol-4-yl]-4-thiazolecarboxamide |
| 3 | N-[5-amino-2-(3(R)-amino-1-piperidinyl)phenyl]-2-[1-(phenylmethyl)-1H- |
| | pyrazol-4-yl]-4-thiazolecarboxamide |
| 4 | N-[5-amino-2-(3(R)-amino-1-piperidinyl)phenyl]-2-[1-[2-(4- |
| | morpholinyl)ethyl]-1H-pyrazol-4-yl]-4-thiazolecarboxamide |
| 5 | 1-[4-amino-2-[[[2-[1-(2-thienylmethyl)-1H-pyrazol-4-yl]-4- |
| | thiazolyl]carbonyl]amino]phenyl]-4-(methylamino)-4-piperidinecarboxamide |
| 6 | N-[2-[4-(aminomethyl)-1-piperidinyl]-3-fluorophenyl]-2-[1-(2-thienylmethyl)- |
| | 1H-pyrazol-4-yl]-4-thiazolecarboxamide |
| 7 | N-[5-amino-2-(2,9-diazaspiro[5.5]undec-9-yl)phenyl]-2-[1-(2-thienylmethyl)- |
| | 1H-pyrazol-4-yl]-4-thiazolecarboxamide |
| 8 | N-[5-amino-2-[4-(aminomethyl)-1-piperidinyl]phenyl]-2-[1-(2-thienylmethyl)- |
| | 1H-pyrazol-4-yl]-4-thiazolecarboxamide |
| 9 | N-[5-amino-2-(3(R)-amino-1-piperidinyl)phenyl]-2-[1-(4-pyridinylmethyl)- |
| | 1H-pyrazol-4-yl]-4-thiazolecarboxamide |
| 10 | N-[5-amino-2-(3(R)-amino-1-pyrrolidinyl)phenyl]-2-[1-(2-thienylmethyl)-1H- |
| | pyrazol-4-yl]-4-thiazolecarboxamide |
| 11 | ethyl 1-[4-amino-2-[[[2-[1-(2-thienylmethyl)-1H-pyrazol-4-yl]-4- |
| | thiazolyl]carbonyl]amino]phenyl]-3-piperidinecarboxylate |
| 12 | ethyl 1-[4-amino-2-[[[2-[1-(2-thienylmethyl)-1H-pyrazol-4-yl]-4- |
| | thiazolyl]carbonyl]amino]phenyl]-4-piperidinecarboxylate |
| 13 | N-[5-amino-2-(3(R)-amino-1-piperidinyl)phenyl]-2-[1-(2-pyridinylmethyl)- |
| | 1H-pyrazol-4-yl]-4-thiazolecarboxamide |
| 14 | N-[5-amino-2-(3(R)-amino-1-piperidinyl)phenyl]-2-[1-[(3,4- |
| | difluorophenyl)methyl]-1H-pyrazol-4-yl]-4-thiazolecarboxamide |
| 15 | N-[5-amino-2-(1,4-dioxa-8-azaspiro[4.5]dec-8-yl)phenyl]-2-[1-[(3- |
| | fluorophenyl)methyl]-1H-pyrazol-4-yl]-4-thiazolecarboxamide |
| 16 | N-[5-amino-2-(3(R)-amino-1-piperidinyl)phenyl]-2-[1-[(3,5- |
| | difluorophenyl)methyl]-1H-pyrazol-4-yl]-4-thiazolecarboxamide |
| 17 | N-[5-amino-2-(3(R)-amino-1-piperidinyl)phenyl]-2-[1-(2-phenylethyl)-1H- |
| | pyrazol-4-yl]-4-thiazolecarboxamide |
| 18 | N-[5-amino-2-(3(R)-amino-1-piperidinyl)phenyl]-2-[1-(3-pyridinylmethyl)- |
| | 1H-pyrazol-4-yl]-4-thiazolecarboxamide |
| 19 | N-[5-amino-2-(3(R)-amino-1-piperidinyl)phenyl]-2-[1-[(3- |
| | fluorophenyl)methyl]-1H-pyrazol-4-yl]-4-thiazolecarboxamide |
| 20 | N-[5-amino-2-(2,9-diazaspiro[5.5]undec-9-yl)phenyl]-2-[1-(2- |
| | pyridinylmethyl)-1H-pyrazol-4-yl]-4-thiazolecarboxamide |
| 21 | N-[5-amino-2-(3(R)-amino-1-piperidinyl)phenyl]-2-[1-[(2- |
| | fluorophenyl)methyl]-1H-pyrazol-4-yl]-4-thiazolecarboxamide |
| 22 | N-[5-amino-2-(3(R)-amino-1-piperidinyl)phenyl]-2-[1-[(2,5- |
| | difluorophenyl)methyl]-1H-pyrazol-4-yl]-4-thiazolecarboxamide |
| 23 | N-[2-(3(R)-amino-1-piperidinyl)-3-fluorophenyl]-2-[1-(2-thienylmethyl)-1H- |
| | pyrazol-4-yl]-4-thiazolecarboxamide |
| 24 | N-[5-amino-2-[4-(aminomethyl)-1-piperidinyl]phenyl]-2-[1-(2- |
| | pyridinylmethyl)-1H-pyrazol-4-yl]-4-thiazolecarboxamide |
| 25 | N-[2-(3(R)-amino-1-piperidinyl)-3-fluorophenyl]-2-[1-(phenylmethyl)-1H- |
| | pyrazol-4-yl]-4-thiazolecarboxamide |
| 26 | N-[5-amino-2-(3(R)-amino-1-piperidinyl)phenyl]-2-[1-(2-methoxyethyl)-1H- |
| | pyrazol-4-yl]-4-thiazolecarboxamide |
| 27 | N-[2-(3(R)-amino-1-piperidinyl)-3-fluorophenyl]-2-[1-(2-pyridinylmethyl)- |
| | 1H-pyrazol-4-yl]-4-thiazolecarboxamide |
| 29 | N-[5-amino-2-(3(R)-amino-1-pyrrolidinyl)phenyl]-2-[1-(2-pyridinylmethyl)- |
| | 1H-pyrazol-4-yl]-4-thiazolecarboxamide |
| 30 | N-[5-amino-2-(3(R)-amino-1-piperidinyl)phenyl]-2-[1-(1-phenylethyl)-1H- |
| | pyrazol-4-yl]-4-thiazolecarboxamide |
| 31 | N-[2-(3(R)-amino-1-piperidinyl)-3-fluorophenyl]-2-[1-(3-pyridinylmethyl)- |
| | 1H-pyrazol-4-yl]-4-thiazolecarboxamide |
| 32 | N-[2-(3(R)-amino-1-piperidinyl)-3-fluorophenyl]-2-[1-[(2,6- |
| | difluorophenyl)methyl]-1H-pyrazol-4-yl]-4-thiazolecarboxamide |
| 33 | N-[2-(3(R)-amino-1-piperidinyl)-3-fluorophenyl]-2-[1-[[2- |
| | (trifluoromethoxy)phenyl]methyl]-1H-pyrazol-4-yl]-4-thiazolecarboxamide |
| 34 | N-[5-amino-2-(1,4-dioxa-8-azaspiro[4.5]dec-8-yl)phenyl]-2-[1-(2- |
| | thienylmethyl)-1H-pyrazol-4-yl]-4-thiazolecarboxamide |
| 35 | N-[2-(3(R)-amino-1-piperidinyl)-3-fluorophenyl]-2-[1-[[2-fluoro-6- |
| | (trifluoromethyl)phenyl]methyl]-1H-pyrazol-4-yl]-4-thiazolecarboxamide |
| 36 | N-[2-(3(R)-amino-1-piperidinyl)-3-fluorophenyl]-2-[1-[[2- |
| | (difluoromethoxy)phenyl]methyl]-1H-pyrazol-4-yl]-4-thiazolecarboxamide |
| 37 | N-[2-(3(R)-amino-1-piperidinyl)-3-fluorophenyl]-2-[1-[[2- |
| | [(trifluoromethyl)thio]phenyl]methyl]-1H-pyrazol-4-yl]-4- |
| | thiazolecarboxamide |
| 38 | N-[2-(3(R)-amino-1-piperidinyl)-3-fluorophenyl]-2-[1-[(2,4,6- |
| | trifluorophenyl)methyl]-1H-pyrazol-4-yl]-4-thiazolecarboxamide |
| 39 | 2-[1-[(2,5-difluorophenyl)methyl]-1H-pyrazol-4-yl]-N-[2-(1- |
| | piperazinyl)phenyl]-4-thiazolecarboxamide |
| 40 | N-[2-(3(R)-amino-1-piperidinyl)-3-fluorophenyl]-2-[1-[2- |
| | (phenylmethoxy)ethyl]-1H-pyrazol-4-yl]-4-thiazolecarboxamide |
| 41 | 2-[1-[2-(4-chlorophenoxy)ethyl]-1H-pyrazol-4-yl]-N-[2-(1- |
| | piperazinyl)phenyl]-4-thiazolecarboxamide |
| 42 | N-[2-(3(R)-amino-1-piperidinyl)-3-fluorophenyl]-2-[1-[[2- |
| | (hydroxymethyl)phenyl]methyl]-1H-pyrazol-4-yl]-4-thiazolecarboxamide |
| 43 | N-[2-(3(R)-amino-1-piperidinyl)-3-fluorophenyl]-2-[1-(3-phenoxypropyl)-1H- |
| | pyrazol-4-yl]-4-thiazolecarboxamide |
| 44 | 2-[[[2-[4-[4-[[[5-amino-2-(3(R)-amino-1-piperidinyl)phenyl]amino]carbonyl]- |
| | 2-thiazolyl]-1H-pyrazol-1-yl]ethyl]amino]carbonyl]benzoic acid |
| 45 | methyl 2-[[4-[4-[[[2-(3(R)-amino-1-piperidinyl)-3- |
| | fluorophenyl]amino]carbonyl]-2-thiazolyl]-1H-pyrazol-1-yl]methyl]-3- |
| | fluorobenzoate |
| 46 | N-[2-(3(R)-amino-1-piperidinyl)-3-fluorophenyl]-2-[1-[[2-fluoro-6- |
| | (hydroxymethyl)phenyl]methyl]-1H-pyrazol-4-yl]-4-thiazolecarboxamide |
| 47 | N-[2-(3(R)-amino-1-piperidinyl)-3-fluorophenyl]-2-[1-(4-piperidinylmethyl)- |
| | 1H-pyrazol-4-yl]-4-thiazolecarboxamide |
| 48 | N-[2-(1-piperazinyl)phenyl]-2-[1-[2-(4-piperidinyl)ethyl]-1H-pyrazol-4-yl]-4- |
| | thiazolecarboxamide |
| 49 | N-[2-(3(R)-amino-1-piperidinyl)-3-fluorophenyl]-2-[1-[(2- |
| | bromophenyl)methyl]-1H-pyrazol-4-yl]-4-thiazolecarboxamide |
| 50 | N-[2-(3(R)-amino-1-piperidinyl)-3-fluorophenyl]-2-[1-[[2-(1h-pyrazol-4- |
| | yl)phenyl]methyl]-1H-pyrazol-4-yl]-4-thiazolecarboxamide |
| 51 | 2-[1-[[2-(aminomethyl)phenyl]methyl]-1H-pyrazol-4-yl]-N-[2-(3(R)-amino-1- |
| | piperidinyl)-3-fluorophenyl]-4-thiazolecarboxamide |
| 52 | N-[2-(3(R)-amino-1-piperidinyl)-3-fluorophenyl]-2-[1-[(2,3- |
| | difluorophenyl)methyl]-1H-pyrazol-4-yl]-4-thiazolecarboxamide |

### Example 3

The assay used to test the compounds' abilities to inhibit phosphorylation of a substrate by PDK1 uses the IMAP® technology system available from Molecular Devices (Silicon Valley, Calif., United States). The technology enables the detection of the phosphorylation of protein substrates by PDK1 and does not require the addition of antibodies to detect substrate phosphorylation. The technology is based on the high-affinity interaction of trivalent metal containing nanoparticles (beads) with phospho-groups on the substrate of interest. The readout for the assay was fluorescence polarization (FP) which increased once the fluorescently labeled substrate was phosphorylated and was bound to the beads as opposed to the unphosphorylated substrate which did not bind the beads and had relatively lower polarization.

In a microwell assay format, the fluorescently-labeled peptide substrate from glycogen synthase-1 (5FAM-PLSRTLSVSSLPGL-NH2 (SEQ ID NO:1) Molecular Devices part no RP7045). was phosphorylated in a kinase reaction. Addition of the IMAP® Binding System (available from Molecular Devices) stopped the kinase reaction and specifically bound the phosphorylated substrates. Phosphorylation and subsequent binding of the substrate to the beads was detected by FP.

The PDK1 IMAP assay utilized recombinant human PDK1 produced in 519 insect cells and containing amino acids 51-556 of the human PDK1 enzyme. The assay measured the change in fluorescence polarization caused by phosphorylation of a peptide substrate by PDK1 Addition of small molecule PDK1 inhibitors results in the reduction of peptide phosphorylation changing the fluorescence polarization which is measured using a fluorescence plate reader. The assay was performed in a 384-well plate with 10 nM PDK1 enzyme, 100 nM peptide substrate 1 (SEQ ID NO:1), 100 nM activated peptide PIFtide and 2.5 uM ATP for 1.5 hours. PIFtide is added separately to the IMAP reaction at 100 nM. The peptide sequence of PIFTtide is RREPRILSEEEQEMFRDFDYIADWC (SEQ ID NO:2). PIFtide is a peptide sequence that interacts with PDK-1 and is derived from PRK2 kinase, a PDK-1 substrate. This sequence is present in the hydrophobic motif present in PDK-1 substrates and binds to the kinase domain of PDK-1. It is thought to act as a docking site for PDK-1 on the substrate and in vitro has been shown enhance PDK-1 phosphorylation of substrates by approximately 4-fold. See Biondi et al., EMBO 19, 979-988 (2000).

The detection beads were then added and allowed to incubate for 1 hour at room temperature and the fluorescence was then read. Staurosporine, a broad spectrum kinase inhibitor, was used as a positive control for the assay resulting in typical IC₅₀s of 3 nM. Test compounds in 100% DMSO at a range of concentrations were added at 0.5 μl 15 minutes prior to ATP addition. The fluorescence polarization units (mP) generated with 1 uM stauroporine is considered to be background mP and the mP units generated with DMSO is considered to be total mP for each assay. The IC₅₀ value is calculated based on fitting the mP units to the total and background mP and the concentration required to inhibit the mP units by 50% is reported to be the IC₅₀.

While this invention has been particularly shown and described with references to embodiments thereof, it will be understood by those skilled in the art that various changes in form and details may be made therein without departing from the meaning of the invention described. Rather, the scope of the invention is defined by the claims that follow.

1. A compound of Formula I: 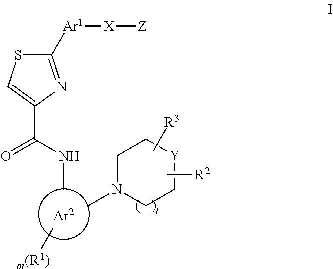
wherein
R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆ alkynyl, halo-C₁-C₆alkyl, C₆-C₁₀aryl, C₃-C₈cycloalkyl, 5- to 10-membered heteroaryl, 5- to 10-membered heterocyclyl, 5- to 10-membered heterocyclenyl, C₆-C₁₀arylC₁-C₆alkyl, C₃-C₈cycloalkylalkyl, 5- to 10-membered heteroarylC₁-C₆alkyl, 5- to 10-membered heterocyclylC₁-C₆alkyl and 5- to 10-membered heterocyclenylC₁-C₆alkyl;
R² and R³ are independently selected from H, OH, halo, C₁-C₆ alkyl, (CR^{a}R^{b})_{q}NR^{b}R⁴, (CR^{a}R^{b})_{q}C(O)OR⁴, (CR^{a}R^{b})_{q}OR⁴, (CR^{a}R^{b})_{q}NR^{b}C(O)R^{a}, (CR^{a}R^{b})_{q}NR^{b}C(O)OR^{a}, (CR^{a}R^{b})_{q}NR^{b}C(O)NR^{a}R^{b} and (CR^{a}R^{b})_{q}C(O)NR^{b}R⁴;
or R² and R³ together form a 5 or 6 membered heterocyclic ring with C, O and N atoms, wherein the heterocyclic ring can be optionally substituted with one or more substituents selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
R^{a} and R^{b} are independently selected from H and C₁-C₆ alkyl;
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})_{q}NR^{a}—, —(CR^{a}R^{b})_{q}O—, —(CR^{a}R^{b})_{q}NR⁴C(O)—, —(CR^{a}R^{b})_{q}NR⁴C(O)NR⁴—, —(CR^{a}R^{b})_{q}NR⁴C(O)O—, —(CR^{a}R^{b})_{q}OC(O)NR⁴—, —(CR^{a}R^{b})_{q}C(O)NR⁴—, —(CR^{a}R^{b})_{q}S(O)₂—, —(CR^{a}R^{b})SO—, —(CR^{a}R^{b})_{q}S(O)₂NR⁴—, —(CR^{a}R^{b})_{q}S(O)₂NR⁴C(O)—, —(CR^{a}R^{b})_{q}C(O)O—, —(CR^{a}R^{b})_{q}OC(O)—, —(CR^{a}R^{b})_{q}OC(O)O—, and —(CR^{a}R^{b})_{q}S—;
Y is C or N;
Z is selected from the group consisting of H, C₁-C₆ alkyl, 5- to 10-membered heteroaryl, 5- to 10-membered heterocyclyl, 5- to 10-membered heterocyclenyl, C₆-C₁₀aryl, C₃-C₈cycloalkyl, wherein said alkyl, cycloalkyl, heteroaryl, heterocyclyl, heterocyclenyl or aryl is optionally substituted with one to three substituents selelected from halo, (CR^{a}R^{b})_{q}OR⁴, —O-haloC₁-C₆alkyl, —S-haloC₁-C₆alkyl, (CR^{a}R^{b})_{q}C(O)OR⁴, —N(R^{a})₂, —(CR^{a}R^{b})_{q}C(O)NHR⁴, —(CR^{a}R^{b})_{q}NR^{a}C(O)R^{a}, CN, C₁-C₆ alkyl, C₁-C₆ alkenyl, C₁-C₆alkynyl, halo-C₁-C₆alkyl, 5- to 10-membered heteroaryl, 5- to 10-membered heterocyclyl, 5- to 10-membered heterocyclenyl and C₆-C₁₀aryl;
m is independently 0, 1, 2, 3 or 4;
n is independently 1, 2 or 3;
t is independently 0 or 1;
q is independently 0, 1, 2 or 3;
or a pharmaceutically acceptable salt thereof.

2. A compound of Formula IIA: 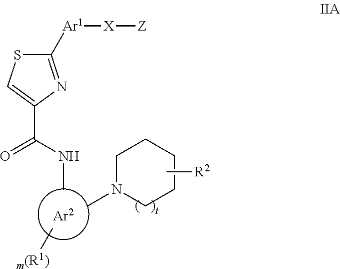
wherein
R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆ alkynyl and halo-C₁-C₆alkyl;
R² is (CR^{a}R^{b})_{q}NHR⁴ or (CR^{a}R^{b})_{q}C(O)OR⁴;
R^{a} and R^{b} are independently selected from H and C₁-C₆ alkyl.
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})ₙO—, —(CR^{a}R^{b})ₙNR^{a}—, —(CR^{a}R^{b})ₙNR⁴C(O)—, —(CR^{a}R^{b})ₙC(O)NR⁴—, and —(CR^{a}R^{b})ₙS—;
Z is selected from the group consisting of C₁-C₆ alkyl, C₆-C₁₀aryl, 5- to 10-membered heteroaryl and 5- to 10-membered heterocyclyl, wherein said alkyl, heteroaryl, heterocyclyl or aryl is optionally substituted with one to three substituents selelected from halo, (CR^{a}R^{b})_{q}OR⁴, —O-haloC₁-C₆alkyl, —S-haloC₁-C₆alkyl, (CR^{a}R^{b})_{q}C(O)OR⁴, —N(R^{a})₂, —(CR^{a}R^{b})_{q}C(O)NHR⁴, —(CR^{a}R^{b})NR^{a}C(O)R^{a}, CN, C₁-C₆ alkyl, C₁-C₆ alkenyl, C₁-C₆alkynyl, halo-C₁-C₆alkyl, 5- to 10-membered heteroaryl, 5- to 10-membered heterocyclyl, 5- to 10-membered heterocyclenyl and C₆-C₁₀aryl;
m is independently 0, 1, 2, 3 or 4;
n is independently 1, 2 or 3;
t is independently 0 or 1;
q is independently 0, 1, 2 or 3;
or a pharmaceutically acceptable salt thereof.

3. The compound of claim 1, wherein Ar² is a 6-membered aryl or heteroaryl.

4. The compound of claim 1, wherein Ar² is phenyl or pyridyl.

5. The compound of claim 1 that is under Formula IIB, 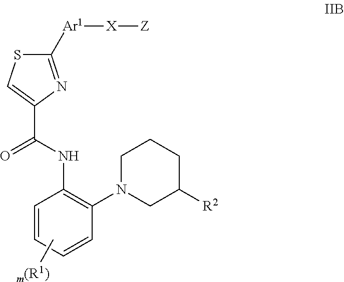
wherein all other substituents are as defined in claim 1.

6. The compound of claim 1 that is under Formula IIC, 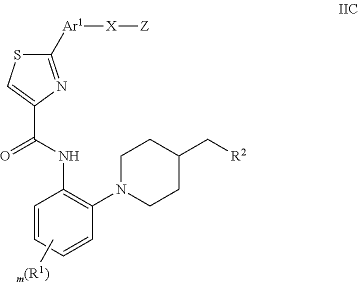
wherein all other substituents are as defined in claim 1.

7. The compound of claim 5, wherein R² is NH₂ or —CH₂—NH₂.

8. The compound of claim 1 that is under Formula IID, 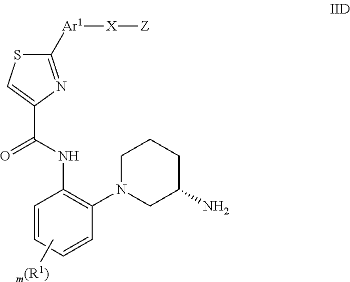
wherein all other substituents are as defined in claim 1.

9. A compound of Formula IIIA: 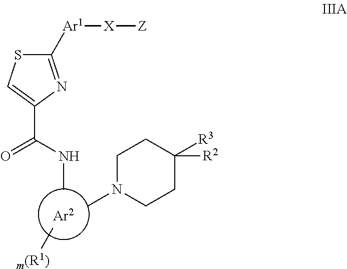
wherein
R¹ is independently selected from the group consisting of halo, OH, (CR^{a}R^{b})_{q}OR⁴, O—C₁-C₆ alkyl, NH₂, CN, C₁-C₆ alkyl, C₁-C₆ alkenyl, C₁-C₆ alkynyl and halo-C₁-C₆alkyl;
R² is (CR^{a}R^{b})_{q}C(O)NHR⁴;
R³ is (CR^{a}R^{b})_{q}NHR⁴;
or R² and R³ together form a 5 or 6 membered heterocyclic ring with C, O and N atoms, wherein the heterocyclic ring can be optionally substituted with one or more substituents selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
R^{a} and R^{b} are independently selected from H and C₁-C₆ alkyl.
R⁴ is independently selected from the group consisting of H, C₁-C₆ alkyl and halo-C₁-C₆alkyl;
Ar¹ is selected from the group consisting of 5-6 membered heteroaryl optionally substituted with one to three substituents of R⁵ selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆ alkynyl, halo-C₁-C₆alkyl, O-halo-C₁-C₆alkyl and S-halo-C₁-C₆alkyl;
Ar² is selected from the group consisting of 5- to 10-membered heteroaryl and C₆-C₁₀aryl;
X is selected from the group consisting of —(CR^{a}R^{b})ₙ—, —(CR^{a}R^{b})ₙO—, —(CR^{a}R^{b})ₙNR^{a}—, —(CR^{a}R^{b})ₙNR⁴C(O)—, —(CR^{a}R^{b})ₙC(O)NR⁴—, and —(CR^{a}R^{b})ₙS—;
Z is selected from the group consisting of C₁-C₆ alkyl, C₆-C₁₀aryl, 5- to 10-membered heteroaryl and 5- to 10-membered heterocyclyl, wherein said alkyl, heteroaryl, heterocyclyl or aryl is optionally substituted with one to three substituents selelected from halo, (CR^{a}R^{b})_{q}OR⁴, —O-halo C₁-C₆alkyl, —S-halo C₁-C₆alkyl, (CR^{a}R^{b})_{q}C(O)OR⁴, —N(R^{a})₂, —(CR^{a}R^{b})_{q}C(O)NHR⁴, —(CR^{a}R^{b})NR^{a}C(O)R^{a}, CN, C₁-C₆ alkyl, C₁-C₆ alkenyl, C₁-C₆alkynyl, halo-C₁-C₆alkyl, 5- to 10-membered heteroaryl, 5- to 10-membered heterocyclyl, 5- to 10-membered heterocyclenyl and C₆-C₁₀aryl;
m is independently 0, 1, 2, 3 or 4;
n is independently 1, 2 or 3;
q is independently 0, 1, 2 or 3;
or a pharmaceutically acceptable salt thereof.

10. The compound of claim 1, wherein Ar¹ is pyrazolyl, optionally substituted with one to three substituents selected from halo, OH, (CR^{a}R^{b})_{q}OR⁴, COOR⁴, O—C₁-C₆ alkyl, NH₂, CN, C₁-C₆ alkyl, C₁-C₆ alkenyl, C₁-C₆alkynyl, halo-C₁-C₆alkyl, O-halo-C₁-C₆alkyl and S-halo-C₁-C₆alkyl.

11. The compound of claim 1, wherein —X—Z is —CH₂-pyridyl, —CH₂-phenyl, —CH₂—CH₂-phenyl or CH₂-thienyl, wherein the pyridyl, phenyl or thienyl is optionally substituted with fluoro.

12. The compound of claim 1 selected from the group consisting of:
N-[5-amino-2-(3(R)-amino-1-piperidinyl)phenyl]-2-[1-[3-(dimethylamino)propyl]-1H-pyrazol-4-yl]-4-thiazolecarboxamide;
N-[5-amino-2-(3(R)-amino-1-piperidinyl)phenyl]-2-[1-(2-thienylmethyl)-1H-pyrazol-4-yl]-4-thiazolecarboxamide;
N-[5-amino-2-(3(R)-amino-1-piperidinyl)phenyl]-2-[1-(phenylmethyl)-1H-pyrazol-4-yl]-4-thiazolecarboxamide;
N-[5-amino-2-(3(R)-amino-1-piperidinyl)phenyl]-2-[1-[2-(4-morpholinyl)ethyl]-1H-pyrazol-4-yl]-4-thiazolecarboxamide;
1-[4-amino-2-[[[2-[1-(2-thienylmethyl)-1H-pyrazol-4-yl]-4-thiazolyl]carbonyl]amino]phenyl]-4-(methylamino)-4-piperidinecarboxamide;
N-[2-[4-(aminomethyl)-1-piperidinyl]-3-fluorophenyl]-2-[1-(2-thienylmethyl)-1H-pyrazol-4-yl]-4-thiazolecarboxamide;
N-[5-amino-2-(2,9-diazaspiro[5.5]undec-9-yl)phenyl]-2-[1-(2-thienylmethyl)-1H-pyrazol-4-yl]-4-thiazolecarboxamide;
N-[5-amino-2-[4-(aminomethyl)-1-piperidinyl]phenyl]-2-[1-(2-thienylmethyl)-1H-pyrazol-4-yl]-4-thiazolecarboxamide;
N-[5-amino-2-(3(R)-amino-1-piperidinyl)phenyl]-2-[1-(4-pyridinylmethyl)-1H-pyrazol-4-yl]-4-thiazolecarboxamide;
N-[5-amino-2-(3(R)-amino-1-pyrrolidinyl)phenyl]-2-[1-(2-thienylmethyl)-1H-pyrazol-4-yl]-4-thiazolecarboxamide;
ethyl 1-[4-amino-2-[[[2-[1-(2-thienylmethyl)-1H-pyrazol-4-yl]-4-thiazolyl]carbonyl]amino]phenyl]-3-piperidinecarboxylate;
ethyl 1-[4-amino-2-[[[2-[1-(2-thienylmethyl)-1H-pyrazol-4-yl]-4-thiazolyl]carbonyl]amino]phenyl]-4-piperidinecarboxylate;
N-[5-amino-2-(3(R)-amino-1-piperidinyl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-[(3,4-difluorophenyl)methyl]-1H-pyrazol-4-yl]-4-thiazolecarboxamide;
N-[5-amino-2-(1,4-dioxa-8-azaspiro[4.5]dec-8-yl)phenyl]-2-[1-[(3-fluorophenyl)methyl]-1H-pyrazol-4-yl]-4-thiazolecarboxamide;
N-[5-amino-2-(3(R)-amino-1-piperidinyl)phenyl]-2-[1-[(3,5-difluorophenyl)methyl]-1H-pyrazol-4-yl]-4-thiazolecarboxamide;
N-[5-amino-2-(3(R)-amino-1-piperidinyl)phenyl]-2-[1-(2-phenylethyl)-1H-pyrazol-4-yl]-4-thiazolecarboxamide;
N-[5-amino-2-(3(R)-amino-1-piperidinyl)phenyl]-2-[1-(3-pyridinylmethyl)-1H-pyrazol-4-yl]-4-thiazolecarboxamide;
N-[5-amino-2-(3(R)-amino-1-piperidinyl)phenyl]-2-[1-[(3-fluorophenyl)methyl]-1H-pyrazol-4-yl]-4-thiazolecarboxamide;
N-[5-amino-2-(2,9-diazaspiro[5.5]undec-9-yl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-[(2-fluorophenyl)methyl]-1H-pyrazol-4-yl]-4-thiazolecarboxamide;
N-[5-amino-2-(3(R)-amino-1-piperidinyl)phenyl]-2-[1-[(2,5-difluorophenyl)methyl]-1H-pyrazol-4-yl]-4-thiazolecarboxamide;
N-[2-(3(R)-amino-1-piperidinyl)-3-fluorophenyl]-2-[1-(2-thienylmethyl)-1H-pyrazol-4-yl]-4-thiazolecarboxamide;
N-[5-amino-2-[4-(aminomethyl)-1-piperidinyl]phenyl]-2-[1-(2-pyridinylmethyl)-1H-pyrazol-4-yl]-4-thiazolecarboxamide;
N-[2-(3(R)-amino-1-piperidinyl)-3-fluorophenyl]-2-[1-(phenylmethyl)-1H-pyrazol-4-yl]-4-thiazolecarboxamide;
N-[5-amino-2-(3(R)-amino-1-piperidinyl)phenyl]-2-[1-(2-methoxyethyl)-1H-pyrazol-4-yl]-4-thiazolecarboxamide;
N-[2-(3(R)-amino-1-piperidinyl)-3-fluorophenyl]-2-[1-(2-pyridinylmethyl)-1H-pyrazol-4-yl]-4-thiazolecarboxamide;
N-[5-amino-2-(3(R)-amino-1-pyrrolidinyl)phenyl]-2-[1-(2-pyridinylmethyl)-1H-pyrazol-4-yl]-4-thiazolecarboxamide;
N-[5-amino-2-(3(R)-amino-1-piperidinyl)phenyl]-2-[1-(1-phenylethyl)-1H-pyrazol-4-yl]-4-thiazolecarboxamide;
N-[2-(3(R)-amino-1-piperidinyl)-3-fluorophenyl]-2-[1-(3-pyridinylmethyl)-1H-pyrazol-4-yl]-4-thiazolecarboxamide;
N-[2-(3(R)-amino-1-piperidinyl)-3-fluorophenyl]-2-[1-[(2,6-difluorophenyl)methyl]-1H-pyrazol-4-yl]-4-thiazolecarboxamide;
N-[2-(3(R)-amino-1-piperidinyl)-3-fluorophenyl]-2-[1-[[2-(trifluoromethoxy)phenyl]methyl]-1H-pyrazol-4-yl]-4-thiazolecarboxamide;
N-[5-amino-2-(1,4-dioxa-8-azaspiro[4.5]dec-8-yl)phenyl]-2-[1-(2-thienylmethyl)-1H-pyrazol-4-yl]-4-thiazolecarboxamide;
N-[2-(3(R)-amino-1-piperidinyl)-3-fluorophenyl]-2-[1-[[2-fluoro-6-(trifluoromethyl)phenyl]methyl]-1H-pyrazol-4-yl]-4-thiazolecarboxamide;
N-[2-(3(R)-amino-1-piperidinyl)-3-fluorophenyl]-2-[1-[[2-(difluoromethoxy)phenyl]methyl]-1H-pyrazol-4-yl]-4-thiazolecarboxamide;
N-[2-(3(R)-amino-1-piperidinyl)-3-fluorophenyl]-2-[1-[[2-[(trifluoromethyl)thio]phenyl]methyl]-1H-pyrazol-4-yl]-4-thiazolecarboxamide;
N-[2-(3(R)-amino-1-piperidinyl)-3-fluorophenyl]-2-[1-[(2,4,6-trifluorophenyl)methyl]-1H-pyrazol-4-yl]-4-thiazolecarboxamide;
2-[1-[(2,5-difluorophenyl)methyl]-1H-pyrazol-4-yl]-N-[2-(1-piperazinyl)phenyl]-4-thiazolecarboxamide;
N-[2-(3(R)-amino-1-piperidinyl)-3-fluorophenyl]-2-[1-[2-(phenylmethoxy)ethyl]-1H-pyrazol-4-yl]-4-thiazolecarboxamide;
2-[1-[2-(4-chlorophenoxy)ethyl]-1H-pyrazol-4-yl]-N-[2-(1-piperazinyl)phenyl]-4-thiazolecarboxamide;
N-[2-(3(R)-amino-1-piperidinyl)-3-fluorophenyl]-2-[1-[[2-(hydroxymethyl)phenyl]methyl]-1H-pyrazol-4-yl]-4-thiazolecarboxamide;
N-[2-(3(R)-amino-1-piperidinyl)-3-fluorophenyl]-2-[1-(3-phenoxypropyl)-1H-pyrazol-4-yl]-4-thiazolecarboxamide;
2-[[[2-[4-[4-[[[5-amino-2-(3(R)-amino-1-piperidinyl)phenyl]amino]carbonyl]-2-thiazolyl]-1H-pyrazol-1-yl]ethyl]amino]carbonyl]benzoic acid;
methyl 2-[[4-[4-[[[2-(3(R)-amino-1-piperidinyl)-3-fluorophenyl]amino]carbonyl]-2-thiazolyl]-1H-pyrazol-1-yl]methyl]-3-fluorobenzoate;
N-[2-(3(R)-amino-1-piperidinyl)-3-fluorophenyl]-2-[1-[[2-fluoro-6-(hydroxymethyl)phenyl]methyl]-1H-pyrazol-4-yl]-4-thiazolecarboxamide;
N-[2-(3(R)-amino-1-piperidinyl)-3-fluorophenyl]-2-[1-(4-piperidinylmethyl)-1H-pyrazol-4-yl]-4-thiazolecarboxamide;
N-[2-(1-piperazinyl)phenyl]-2-[1-[2-(4-piperidinyl)ethyl]-1H-pyrazol-4-yl]-4-thiazolecarboxamide;
N-[2-(3(R)-amino-1-piperidinyl)-3-fluorophenyl]-2-[1-[(2-bromophenyl)methyl]-1H-pyrazol-4-yl]-4-thiazolecarboxamide;
N-[2-(3(R)-amino-1-piperidinyl)-3-fluorophenyl]-2-[1-[[2-(1h-pyrazol-4-yl)phenyl]methyl]-1H-pyrazol-4-yl]-4-thiazolecarboxamide;
2-[1-[[2-(aminomethyl)phenyl]methyl]-1H-pyrazol-4-yl]-N-[2-(3(R)-amino-1-piperidinyl)-3-fluorophenyl]-4-thiazolecarboxamide;
N-[2-(3(R)-amino-1-piperidinyl)-3-fluorophenyl]-2-[1-[(2,3-difluorophenyl)methyl]-1H-pyrazol-4-yl]-4-thiazolecarboxamide;
or a stereoisomer thereof;
or a pharmaceutically acceptable salt thereof;
or a pharmaceutically acceptable salt of the stereoisomer thereof.

13. A pharmaceutical composition comprising a therapeutically effective amount of the compound of claim 1 and a pharmaceutically acceptable carrier and optionally other therapeutic ingredients.

14. Use of a compound according to claim 1 for the preparation of a medicament for the treatment of cancer.
